SEQUENCE LISTING (84 sequences)

SEQ ID NO: 1 - protein, 468 aa, Clostridium saccharoperbutylacetonicum
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPGMVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYSINKKWVGKDAKLFLDEIDVESPSSVKCIICEVSARHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG

SEQ ID NO: 2 - DNA, 1407 bp, Clostridium saccharoperbutylacetonicum
atgattaaagacacgctagtttctataacaaaagatttaaaattaaaaacaaatgttgaaaatgccaatctaaagaactacaaggatgattcttcatgtttcggagttttcgaaaatgttgaaaatgctataagcaatgccgtacacgcacaaaagatattatcccttcattatacaaaagaacaaagagaaaaaatcataactgagataagaaaggccgcattagaaaataaagagattctagctacaatgattcttgaagaaacacatatgggaagatatgaagataaaatattaaagcatgaattagtagctaaatacactcctgggacagaagatttaactactactgcttggtcaggagataacgggcttacagttgtagaaatgtctccatatggcgttataggtgcaataactccttctacgaatccaactgaaactgtaatatgtaatagtataggcatgatagctgctggaaatactgtggtatttaacggacatccaggcgctaaaaaatgtgttgcttttgctgtcgaaatgataaataaagctattatttcatgtggtggtcctgagaatttagtaacaactataaaaaatccaactatggactctctagatgcaattattaagcacccttcaataaaactactttgcggaactggagggccaggaatggtaaaaaccctcttaaattctggtaagaaagctataggtgctggtgctggaaatccaccagttattgtagatgatactgctgatatagaaaaggctggtaagagtatcattgaaggctgttcttttgataataatttaccttgtattgcagaaaaagaagtatttgtttttgagaacgttgcagatgatttaatatctaacatgctaaaaaataatgctgtaattataaatgaagatcaagtatcaaagttaatagatttagtattacaaaaaaataatgaaactcaagaatactctataaataagaaatgggtcggaaaagatgcaaaattattcttagatgaaatagatgttgagtctccttcaagtgttaaatgcataatctgcgaagtaagtgcaaggcatccatttgttatgacagaactcatgatgccaatattaccaattgtaagagttaaagatatagatgaagctattgaatatgcaaaaatagcagaacaaaatagaaaacatagtgcctatatttattcaaaaaatatagacaacctaaataggtttgaaagagaaatcgatactactatctttgtaaagaatgctaaatcttttgccggtgttggttatgaagcagaaggctttacaactttcactattgctggatccactggtgaaggaataacttctgcaagaaattttacaagacaaagaagatgtgtactcgccggttaa

SEQ ID NO: 3 - protein, 468 aa, Artificial Sequence
  Synthetic (bldH)
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPGIVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYSINKKWVGKDAKLFLDEIDVESPSSVKCIICEVSARHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG

SEQ ID NO: 4 - protein, 468 aa, Artificial Sequence
  Synthetic (BldI)
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPGLVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYSINKKWVGKDAKLFLDEIDVESPSSVKCIICEVSARHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG

SEQ ID NO: 5 - protein, 468 aa, Artificial Sequence
  Synthetic (BldJ)
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPGQVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYSINKKWVGKDAKLFLDEIDVESPSSVKCIICEVSARHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG

SEQ ID NO: 6 - protein, 468 aa, Artificial Sequence
  Synthetic (BldL)
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPGVVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYSINKKWVGKDAKLFLDEIDVESPSSVKCIICEVSARHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG

SEQ ID NO: 7 - protein, 468 aa, Artificial Sequence
  Synthetic (BldS2)
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPGMVKTLLNSGKKAIGAGAGNPPVIVDDTADIEKAGKSIIEGCSFDNNIPCIAEKEVFVFENVADDLISNMLKNNAVIINEDQVSKLIDLVLQKNNETQEYSINKKWVGKDAKLFLDEIDVESPSSVKCIICEVSARHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG

SEQ ID NO: 8 - protein, 468 aa, Artificial Sequence
  Synthetic (BldS)
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPGLVKTLLNSGKKAIGAGAGNPPVIVDDTADIEKAGKSIIEGCSFDNNIPCIAEKEVFVFENVADDLISNMLKNNAVIINEDQVSKLIDLVLQKNNETQEYSINKKWVGKDAKLFLDEIDVESPSSVKCIICEVSARHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG

SEQ ID NO: 9 - protein, 468 aa, Artificial Sequence
  Synthetic (Bld(G226I))
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPIMVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYSINKKWVGKDAKLFLDEIDVESPSSVKCIICEVSARHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG

SEQ ID NO: 10 - DNA, 1407 bp, Artificial Sequence
  Synthetic (bldH)
atgattaaagacacgctagtttctataacaaaagatttaaaattaaaaacaaatgttgaaaatgccaatctaaagaactacaaggatgattcttcatgtttcggagttttcgaaaatgttgaaaatgctataagcaatgccgtacacgcacaaaagatattatcccttcattatacaaaagaacaaagagaaaaaatcataactgagataagaaaggccgcattagaaaataaagagattctagctacaatgattcttgaagaaacacatatgggaagatatgaagataaaatattaaagcatgaattagtagctaaatacactcctgggacagaagatttaactactactgcttggtcaggagataacgggcttacagttgtagaaatgtctccatatggcgttataggtgcaataactccttctacgaatccaactgaaactgtaatatgtaatagtataggcatgatagctgctggaaatactgtggtatttaacggacatccaggcgctaaaaaatgtgttgcttttgctgtcgaaatgataaataaagctattatttcatgtggtggtcctgagaatttagtaacaactataaaaaatccaactatggactctctagatgcaattattaagcacccttcaataaaactactttgcggaactggagggccaggaatcgtaaaaaccctcttaaattctggtaagaaagctataggtgctggtgctggaaatccaccagttattgtagatgatactgctgatatagaaaaggctggtaagagtatcattgaaggctgttcttttgataataatttaccttgtattgcagaaaaagaagtatttgtttttgagaacgttgcagatgatttaatatctaacatgctaaaaaataatgctgtaattataaatgaagatcaagtatcaaagttaatagatttagtattacaaaaaaataatgaaactcaagaatactctataaataagaaatgggtcggaaaagatgcaaaattattcttagatgaaatagatgttgagtctccttcaagtgttaaatgcataatctgcgaagtaagtgcaaggcatccatttgttatgacagaactcatgatgccaatattaccaattgtaagagttaaagatatagatgaagctattgaatatgcaaaaatagcagaacaaaatagaaaacatagtgcctatatttattcaaaaaatatagacaacctaaataggtttgaaagagaaatcgatactactatctttgtaaagaatgctaaatcttttgccggtgttggttatgaagcagaaggctttacaactttcactattgctggatccactggtgaaggaataacttctgcaagaaattttacaagacaaagaagatgtgtactcgccggttaa

SEQ ID NO: 11 - DNA, 1407 bp, Artificial Sequence
  Synthetic (bldI)
atgattaaagacacgctagtttctataacaaaagatttaaaattaaaaacaaatgttgaaaatgccaatctaaagaactacaaggatgattcttcatgtttcggagttttcgaaaatgttgaaaatgctataagcaatgccgtacacgcacaaaagatattatcccttcattatacaaaagaacaaagagaaaaaatcataactgagataagaaaggccgcattagaaaataaagagattctagctacaatgattcttgaagaaacacatatgggaagatatgaagataaaatattaaagcatgaattagtagctaaatacactcctgggacagaagatttaactactactgcttggtcaggagataacgggcttacagttgtagaaatgtctccatatggcgttataggtgcaataactccttctacgaatccaactgaaactgtaatatgtaatagtataggcatgatagctgctggaaatactgtggtatttaacggacatccaggcgctaaaaaatgtgttgcttttgctgtcgaaatgataaataaagctattatttcatgtggtggtcctgagaatttagtaacaactataaaaaatccaactatggactctctagatgcaattattaagcacccttcaataaaactactttgcggaactggagggccaggactcgtaaaaaccctcttaaattctggtaagaaagctataggtgctggtgctggaaatccaccagttattgtagatgatactgctgatatagaaaaggctggtaagagtatcattgaaggctgttcttttgataataatttaccttgtattgcagaaaaagaagtatttgtttttgagaacgttgcagatgatttaatatctaacatgctaaaaaataatgctgtaattataaatgaagatcaagtatcaaagttaatagatttagtattacaaaaaaataatgaaactcaagaatactctataaataagaaatgggtcggaaaagatgcaaaattattcttagatgaaatagatgttgagtctccttcaagtgttaaatgcataatctgcgaagtaagtgcaaggcatccatttgttatgacagaactcatgatgccaatattaccaattgtaagagttaaagatatagatgaagctattgaatatgcaaaaatagcagaacaaaatagaaaacatagtgcctatatttattcaaaaaatatagacaacctaaataggtttgaaagagaaatcgatactactatctttgtaaagaatgctaaatcttttgccggtgttggttatgaagcagaaggctttacaactttcactattgctggatccactggtgaaggaataacttctgcaagaaattttacaagacaaagaagatgtgtactcgccggttaa

SEQ ID NO: 12 - DNA, 1407 bp, Artificial Sequence
  Synthetic (bld J)
atgattaaagacacgctagtttctataacaaaagatttaaaattaaaaacaaatgttgaaaatgccaatctaaagaactacaaggatgattcttcatgtttcggagttttcgaaaatgttgaaaatgctataagcaatgccgtacacgcacaaaagatattatcccttcattatacaaaagaacaaagagaaaaaatcataactgagataagaaaggccgcattagaaaataaagagattctagctacaatgattcttgaagaaacacatatgggaagatatgaagataaaatattaaagcatgaattagtagctaaatacactcctgggacagaagatttaactactactgcttggtcaggagataacgggcttacagttgtagaaatgtctccatatggcgttataggtgcaataactccttctacgaatccaactgaaactgtaatatgtaatagtataggcatgatagctgctggaaatactgtggtatttaacggacatccaggcgctaaaaaatgtgttgcttttgctgtcgaaatgataaataaagctattatttcatgtggtggtcctgagaatttagtaacaactataaaaaatccaactatggactctctagatgcaattattaagcacccttcaataaaactactttgcggaactggagggccaggacaggtaaaaaccctcttaaattctggtaagaaagctataggtgctggtgctggaaatccaccagttattgtagatgatactgctgatatagaaaaggctggtaagagtatcattgaaggctgttcttttgataataatttaccttgtattgcagaaaaagaagtatttgtttttgagaacgttgcagatgatttaatatctaacatgctaaaaaataatgctgtaattataaatgaagatcaagtatcaaagttaatagatttagtattacaaaaaaataatgaaactcaagaatactctataaataagaaatgggtcggaaaagatgcaaaattattcttagatgaaatagatgttgagtctccttcaagtgttaaatgcataatctgcgaagtaagtgcaaggcatccatttgttatgacagaactcatgatgccaatattaccaattgtaagagttaaagatatagatgaagctattgaatatgcaaaaatagcagaacaaaatagaaaacatagtgcctatatttattcaaaaaatatagacaacctaaataggtttgaaagagaaatcgatactactatctttgtaaagaatgctaaatcttttgccggtgttggttatgaagcagaaggctttacaactttcactattgctggatccactggtgaaggaataacttctgcaagaaattttacaagacaaagaagatgtgtactcgccggttaa

SEQ ID NO: 13 - DNA, 1407 bp, Artificial Sequence
  Synthetic (bld L)
atgattaaagacacgctagtttctataacaaaagatttaaaattaaaaacaaatgttgaaaatgccaatctaaagaactacaaggatgattcttcatgtttcggagttttcgaaaatgttgaaaatgctataagcaatgccgtacacgcacaaaagatattatcccttcattatacaaaagaacaaagagaaaaaatcataactgagataagaaaggccgcattagaaaataaagagattctagctacaatgattcttgaagaaacacatatgggaagatatgaagataaaatattaaagcatgaattagtagctaaatacactcctgggacagaagatttaactactactgcttggtcaggagataacgggcttacagttgtagaaatgtctccatatggcgttataggtgcaataactccttctacgaatccaactgaaactgtaatatgtaatagtataggcatgatagctgctggaaatactgtggtatttaacggacatccaggcgctaaaaaatgtgttgcttttgctgtcgaaatgataaataaagctattatttcatgtggtggtcctgagaatttagtaacaactataaaaaatccaactatggactctctagatgcaattattaagcacccttcaataaaactactttgcggaactggagggccaggagtcgtaaaaaccctcttaaattctggtaagaaagctataggtgctggtgctggaaatccaccagttattgtagatgatactgctgatatagaaaaggctggtaagagtatcattgaaggctgttcttttgataataatttaccttgtattgcagaaaaagaagtatttgtttttgagaacgttgcagatgatttaatatctaacatgctaaaaaataatgctgtaattataaatgaagatcaagtatcaaagttaatagatttagtattacaaaaaaataatgaaactcaagaatactctataaataagaaatgggtcggaaaagatgcaaaattattcttagatgaaatagatgttgagtctccttcaagtgttaaatgcataatctgcgaagtaagtgcaaggcatccatttgttatgacagaactcatgatgccaatattaccaattgtaagagttaaagatatagatgaagctattgaatatgcaaaaatagcagaacaaaatagaaaacatagtgcctatatttattcaaaaaatatagacaacctaaataggtttgaaagagaaatcgatactactatctttgtaaagaatgctaaatcttttgccggtgttggttatgaagcagaaggctttacaactttcactattgctggatccactggtgaaggaataacttctgcaagaaattttacaagacaaagaagatgtgtactcgccggttaa

SEQ ID NO: 14 - DNA, 1407 bp, Artificial Sequence
  Synthetic (bldS2)
atgattaaagacacgctagtttctataacaaaagatttaaaattaaaaacaaatgttgaaaatgccaatctaaagaactacaaggatgattcttcatgtttcggagttttcgaaaatgttgaaaatgctataagcaatgccgtacacgcacaaaagatattatcccttcattatacaaaagaacaaagagaaaaaatcataactgagataagaaaggccgcattagaaaataaagagattctagctacaatgattcttgaagaaacacatatgggaagatatgaagataaaatattaaagcatgaattagtagctaaatacactcctgggacagaagatttaactactactgcttggtcaggagataacgggcttacagttgtagaaatgtctccatatggcgttataggtgcaataactccttctacgaatccaactgaaactgtaatatgtaatagtataggcatgatagctgctggaaatactgtggtatttaacggacatccaggcgctaaaaaatgtgttgcttttgctgtcgaaatgataaataaagctattatttcatgtggtggtcctgagaatttagtaacaactataaaaaatccaactatggactctctagatgcaattattaagcacccttcaataaaactactttgcggaactggagggccaggaatggtaaaaaccctcttaaattctggtaagaaagctataggtgctggtgctggaaatccaccagttattgtagatgatactgctgatatagaaaaggctggtaagagtatcattgaaggctgttcttttgataataatatcccttgtattgcagaaaaagaagtatttgtttttgagaacgttgcagatgatttaatatctaacatgctaaaaaataatgctgtaattataaatgaagatcaagtatcaaagttaatagatttagtattacaaaaaaataatgaaactcaagaatactctataaataagaaatgggtcggaaaagatgcaaaattattcttagatgaaatagatgttgagtctccttcaagtgttaaatgcataatctgcgaagtaagtgcaaggcatccatttgttatgacagaactcatgatgccaatattaccaattgtaagagttaaagatatagatgaagctattgaatatgcaaaaatagcagaacaaaatagaaaacatagtgcctatatttattcaaaaaatatagacaacctaaataggtttgaaagagaaatcgatactactatctttgtaaagaatgctaaatcttttgccggtgttggttatgaagcagaaggctttacaactttcactattgctggatccactggtgaaggaataacttctgcaagaaattttacaagacaaagaagatgtgtactcgccggttaa

SEQ ID NO: 15 - DNA, 1407 bp, Artificial Sequence
  Synthetic (bldS)
atgattaaagacacgctagtttctataacaaaagatttaaaattaaaaacaaatgttgaaaatgccaatctaaagaactacaaggatgattcttcatgtttcggagttttcgaaaatgttgaaaatgctataagcaatgccgtacacgcacaaaagatattatcccttcattatacaaaagaacaaagagaaaaaatcataactgagataagaaaggccgcattagaaaataaagagattctagctacaatgattcttgaagaaacacatatgggaagatatgaagataaaatattaaagcatgaattagtagctaaatacactcctgggacagaagatttaactactactgcttggtcaggagataacgggcttacagttgtagaaatgtctccatatggcgttataggtgcaataactccttctacgaatccaactgaaactgtaatatgtaatagtataggcatgatagctgctggaaatactgtggtatttaacggacatccaggcgctaaaaaatgtgttgcttttgctgtcgaaatgataaataaagctattatttcatgtggtggtcctgagaatttagtaacaactataaaaaatccaactatggactctctagatgcaattattaagcacccttcaataaaactactttgcggaactggagggccaggactcgtaaaaaccctcttaaattctggtaagaaagctataggtgctggtgctggaaatccaccagttattgtagatgatactgctgatatagaaaaggctggtaagagtatcattgaaggctgttcttttgataataatatcccttgtattgcagaaaaagaagtatttgtttttgagaacgttgcagatgatttaatatctaacatgctaaaaaataatgctgtaattataaatgaagatcaagtatcaaagttaatagatttagtattacaaaaaaataatgaaactcaagaatactctataaataagaaatgggtcggaaaagatgcaaaattattcttagatgaaatagatgttgagtctccttcaagtgttaaatgcataatctgcgaagtaagtgcaaggcatccatttgttatgacagaactcatgatgccaatattaccaattgtaagagttaaagatatagatgaagctattgaatatgcaaaaatagcagaacaaaatagaaaacatagtgcctatatttattcaaaaaatatagacaacctaaataggtttgaaagagaaatcgatactactatctttgtaaagaatgctaaatcttttgccggtgttggttatgaagcagaaggctttacaactttcactattgctggatccactggtgaaggaataacttctgcaagaaattttacaagacaaagaagatgtgtactcgccggttaa

SEQ ID NO: 16 - DNA, 1418 bp, Artificial Sequence
  Synthetic (bld(G226I))
tgattaaagacacgctagtttctataacaaaagatttaaaattaaaaacaaatgttgaaaatgccaatctaaagaactacaaggatgattcttcatgtttcggagttttcgaaaatgttgaaaatgctataagcaatgccgtacacgcacaaaagatattatcccttcattatacaaaagaacaaagagaaaaaatcataactgagataagaaaggccgcattagaaaataaagagattctagctacaatgattcttgaagaaacacatatgggaagatatgaagataaaatattaaagcatgaattagtagctaaatacactcctgggacagaagatttaactactactgcttggtcaggagataacgggcttacagttgtagaaatgtctccatatggcgttataggtgcaataactccttctacgaatccaactgaaactgtaatatgtaatagtataggcatgatagctgctggaaatactgtggtatttaacggacatccaggcgctaaaaaatgtgttgcttttgctgtcgaaatgataaataaagctattatttcatgtggtggtcctgagaatttagtaacaactataaaaaatccaactatggactctctagatgcaattattaagcacccttcaataaaactactttgcggaactggagggccactaatggtaaaaaccctcttaaatggccactaatggtctggtaagaaagctataggtgctggtgctggaaatccaccagttattgtagatgatactgctgatatagaaaaggctggtaagagtatcattgaaggctgttcttttgataataatttaccttgtattgcagaaaaagaagtatttgtttttgagaacgttgcagatgatttaatatctaacatgctaaaaaataatgctgtaattataaatgaagatcaagtatcaaagttaatagatttagtattacaaaaaaataatgaaactcaagaatactctataaataagaaatgggtcggaaaagatgcaaaattattcttagatgaaatagatgttgagtctccttcaagtgttaaatgcataatctgcgaagtaagtgcaaggcatccatttgttatgacagaactcatgatgccaatattaccaattgtaagagttaaagatatagatgaagctattgaatatgcaaaaatagcagaacaaaatagaaaacatagtgcctatatttattcaaaaaatatagacaacctaaataggtttgaaagagaaatcgatactactatctttgtaaagaatgctaaatcttttgccggtgttggttatgaagcagaaggctttacaactttcactattgctggatccactggtgaaggaataacttctgcaagaaattttacaagacaaagaagatgtgtactcgccggttaa

SEQ ID NO: 17 - DNA, 1296 bp, Porphyromonas gingivalis
atgaaagacgtgttagcggaatatgcctcccgaattgtttcggccgaagaggcagtcaaacatatcaaaaatggagagcgtgtcgctttatcacatgctgccggagttcctcagagttgtgttgacgcactggtgcaacaggcggacctgtttcagaatgtggagatttaccacatgctgtgtctcggcgaaggaaaatatatggcacctgaaatggcccctcacttccggcacataaccaattttgttggtggtaactctcgtaaagcagtggaggaaaatagagccgacttcattccggtattcttttatgaagtgccatcaatgattcggaaagatatccttcatatagatgtggccattgtccaactctcaatgccagatgagaatggttactgcagctttggcgtatcttgcgattatagcaaaccggcggcggaatcggcgcatttagttattggggaaatcaaccgtcagatgccatatgtgcatggtgacaacttgattcacatatcgaagttggattacatcgtgatggcggattacccaatttattctctggcgaagcccaaaatcggagaagtagaggaagctatcggccgtaactgtgccgagcttattgaagatggtgccaccctacagctgggtatcggcgcgattccggatgcagctctgctgtttctgaaggacaaaaaagatctggggattcatactgaaatgttctccgatggcgttgttgaactggtgcgcagtggtgtaattactggaaaaaaaaagacattgcatcccggtaagatggtcgcgacgtttcttatgggatcagaagacgtgtatcatttcatcgacaagaatccggatgtggaactgtatccggttgattacgtcaatgatccgagggttatcgctcagaatgataatatggtcagcatcaatagctgtatcgagatcgatctaatgggccaagtggtgagcgagtgcataggctccaaacagtttagtggcaccgggggtcaagtagattatgtccgcggggcagcttggtctaaaaacggcaaaagcatcatggcaattccctcaacagccaaaaacggtactgcatctcggatagttcctataattgcagagggcgctgctgtaacaaccctccgcaacgaagtcgactacgttgttacggaatatgggatagcacagttaaaaggtaagagtttgcgtcagcgcgcagaagctcttattgcgatagcccacccggactttagagaggaactgacgaagcatctgcgcaaacgttttggttaa

SEQ ID NO: 18 - protein, 451 aa, Clostridium kluyveri
MEIKEMVSLARKAQKEYQATHNQEAVDNICRAAAKVIYENAAILAREAVDETGMGVYEHKVAKNQGKSKGVWYNLHNKKSIGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAYSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 19 - DNA, 1356 bp, Clostridium kluyveri
atggaaataaaagagatggtgtcgttggcaaggaaagctcagaaggaatatcaagcgacccataatcaagaagcagttgataacatttgccgagctgcagcaaaagtgatttatgaaaatgcagctatactggctcgcgaagcagtagacgaaaccggcatgggcgtatatgaacataaagtggccaagaatcaggggaaatccaaaggcgtctggtacaatttgcacaataaaaaatcgatcggtatcttaaatatagacgagagaaccgggatgatcgagatagcaaaacctatcggggttgttggagccgtaaccccgacgacaaacccgattgtgactccaatgagcaacatcatttttgcccttaagacatgcaatgccattattatcgccccacatcccagatccaaaaaatgctcagcacatgcagttcgtctgataaaggaagcaatcgctccgtttaatgtcccggagggaatggttcagatcattgaagagcccagcatcgagaaaactcaggaactaatgggcgccgtggatgtggtagttgcgacgggtggtatgggtatggtgaaatctgcatattcttcagggaagccttcttttggtgtaggagccggtaacgttcaagtgatcgtggatagtaatatcgattttgaagctgcggcagaaaaaattatcaccggccgtgctttcgacaatgggatcatctgttcaggcgaacagagtatcatctacaacgaagctgacaaggaagctgtcttcacagccttccgcaaccatggtgcatatttttgtgatgaagcggagggagatcgggcccgtgctgcgatttttgagaatggcgccatcgcgaaagatgtagtcggccagagcgttgcctttatcgcgaagaaagcaaatatcaatataccggagggtacccgtattctggttgttgaagctcgcggcgtcggagcagaggatgtcatatgtaaggaaaaaatgtgtccagttatgtgcgccttaagctacaagcacttcgaggaaggtgtagaaatcgcacgtacgaacttggccaacgaaggtaacggccatacctgtgcgatccattccaacaatcaggcgcatatcatactggcaggttcagaactgacggtttcgcggatcgtggtcaatgcgccgagtgccactacagcaggcggtcacatccaaaatggtctggcagtgacaaatacgctcggatgcgggagttggggtaataactctatctccgagaactttacttataaacacctgttaaacattagccgcatagcgccgcttaattcaagcattcacattcctgatgacaaagagatctgggaactctaa

SEQ ID NO: 20 - protein, 1214 aa, Mycobacterium bovis
MYRKFRDDPSSVDPSWHEFLVDYSPEPTSQPAAEPTRVTSPLVAERAAAAAPQAPPKPADTAAAGNGVVAALAAKTAVPPPAEGDEVAVLRGAAAAVVKNMSASLEVPTATSVRAVPAKLLIDNRIVINNQLKRTRGGKISFTHLLGYALVQAVKKFPNMNRHYTEVDGKPTAVTPAHTNLGLAIDLQGKDGKRSLVVAGIKRCETMRFAQFVTAYEDIVRRARDGKLTTEDFAGVTISLTNPGTIGTVHSVPRLMPGQGAIIGVGAMEYPAEFQGASEERIAELGIGKLITLTSTYDHRIIQGAESGDFLRTIHELLLSDGFWDEVFRELSIPYLPVRWSTDNPDSIVDKNARVMNLIAAYRNRGHLMADTDPLRLDKARFRSHPDLEVLTHGLTLWDLDRVFKVDGFAGAQYKKLRDVLGLLRDAYCRHIGVEYAHILDPEQKEWLEQRVETKHVKPTVAQQKYILSKLNAAEAFETFLQTKYVGQKRFSLEGAESVIPMMDAAIDQCAEHGLDEVVIGMPHRGRLNVLANIVGKPYSQIFTEFEGNLNPSQAHGSGDVKYHLGATGLYLQMFGDNDIQVSLTANPSHLEAVDPVLEGLVRAKQDLLDHGSIDSDGQRAFSVVPLMLHGDAAFAGQGVVAETLNLANLPGYRVGGTIHIIVNNQIGFTTAPEYSRSSEYCTDVAKMIGAPIFHVNGDDPEACVWVARLAVDFRQRFKKDVVIDMLCYRRRGHNEGDDPSMTNPYMYDVVDTKRGARKSYTEALIGRGDISMKEAEDALRDYQGQLERVFNEVRELEKHGVQPSESVESDQMIPAGLATAVDKSLLARIGDAFLALPNGFTAHPRVQPVLEKRREMAYEGKIDWAFGELLALGSLVAEGKLVRLSGQDSRRGTFSQRHSVLIDRHTGEEFTPLQLLATNSDGSPTGGKFLVYDSPLSEYAAVGFEYGYTVGNPDAVVLWEAQFGDFVNGAQSIIDEFISSGEAKWGQLSNVVLLLPHGHEGQGPDHTSARIERFLQLWAEGSMTIAMPSTPSNYFHLLRRHALDGIQRPLIVFTPKSMLRHKAAVSEIKDFTEIKFRSVLEEPTYEDGIGDRNKVSRILLTSGKLYYELAARKAKDNRNDLAIVRLEQLAPLPRRRLRETLDRYENVKEFFWVQEEPANQGAWPRFGLELPELLPDKLAGIKRISRRAMSAPSSGSSKVHAVEQQEILDEAFG

SEQ ID NO: 21 - DNA, 3645 bp, Mycobacterium bovis
atgtaccgtaaattccgtgatgacccgtcttctgttgatccgtcttggcacgaatttctggtcgattactccccggaaccaacttcccagccggccgctgaaccgacccgcgttacgtcccctctggtcgcggaacgtgcagctgcggcagcaccgcaggcgccaccaaaacctgctgataccgctgcagctggtaatggtgtggttgctgcactggctgctaaaacggctgttccgccgcctgctgaaggtgatgaagtggccgtgctgcgtggtgcggcagccgcggtcgtcaaaaacatgagcgcgtctctggaagtgccgacggcgaccagcgtgcgcgcggttccagcgaaactgctgattgataatcgtattgtgatcaacaaccagctgaaacgtacccgtggtggcaaaattagctttacccacctgctgggttatgccctggtgcaggcggtgaagaaattcccgaacatgaaccgtcactacaccgaagtcgacggtaaaccgactgccgtgaccccggcacacaccaacctgggcctggcaattgacctgcagggcaaggatggcaagcgttccctggtagtagctggtattaaacgttgcgaaaccatgcgctttgcacagttcgtaaccgcgtacgaagatatcgtacgtcgcgcacgtgatggcaaactgactaccgaagacttcgcgggtgtgaccatttccctgaccaacccgggcaccatcggtactgtacatagcgtaccacgtctgatgccgggtcagggtgcgattatcggcgttggtgctatggagtatccggccgagtttcagggtgcttccgaagagcgtatcgcggaactgggtattggtaaactgattaccctgacgagcacctacgaccaccgcatcatccagggcgccgaaagcggtgacttcctgcgtaccatccatgaactgctgctgtccgatggtttctgggatgaagtcttccgcgaactgtctattccgtacctgccggtccgttggtccaccgataacccggattctattgtagacaaaaacgcccgcgttatgaacctgatcgcagcgtatcgtaatcgtggccacctgatggcagacacggaccctctgcgtctggacaaagcgcgttttcgcagccacccggacctggaagttctgactcatggcctgactctgtgggatctggatcgcgtatttaaagtggatggctttgcaggtgcccagtacaagaaactgcgtgatgttctgggcctgctgcgtgacgcctattgccgccatattggtgttgaatacgcgcacatcctggacccagagcagaaagaatggctggagcagcgtgtggaaaccaaacacgttaagccgaccgtagcgcagcagaaatacatcctgtctaagctgaacgctgccgaggctttcgaaacctttctgcagacgaaatatgttggtcagaaacgcttctccctggagggtgcagaatctgtgatcccgatgatggatgctgcgatcgaccagtgcgctgaacacggcctggacgaggtagtgatcggtatgccgcaccgtggccgtctgaacgttctggctaacatcgttggtaaaccgtacagccagatctttactgaattcgaaggcaacctgaacccgtcccaggctcatggttccggcgacgtgaaataccatctgggcgcaactggtctgtacctgcagatgttcggtgataatgacatccaggtatctctgaccgctaatccgtcccacctggaagcggttgacccggtactggaaggcctggttcgtgcaaaacaagatctgctggaccacggtagcatcgattctgacggtcagcgtgccttctctgtggttccgctgatgctgcacggcgatgcggcttttgcaggccagggtgttgttgctgaaacgctgaacctggcgaacctgccgggctaccgtgttggtggcactatccatatcatcgttaacaaccagatcggcttcacgaccgcgccggaatactctcgctctagcgaatactgcactgatgtggctaagatgattggcgccccaatcttccacgttaacggtgacgacccggaagcgtgtgtgtgggttgcccgtctggctgtggatttccgtcaacgtttcaaaaaggacgttgttatcgacatgctgtgttaccgtcgtcgcggccacaacgaaggcgacgatccgagcatgactaacccttacatgtacgatgtagttgacaccaaacgtggcgcacgtaaaagctatactgaagcgctgatcggtcgtggtgatatctctatgaaagaagcagaagacgcactgcgcgactatcaaggccaactggaacgcgttttcaacgaagttcgcgagctggagaaacacggtgtccaacctagcgaatctgtggaatctgaccagatgatcccggcgggtctggcaactgcagtggacaaaagcctgctggcacgtattggcgacgcgttcctggctctgccgaacggtttcactgcacacccacgtgtacagccggttctggaaaaacgtcgtgaaatggcctacgaaggtaaaatcgactgggcttttggtgagctgctggcgctgggctccctggttgcggagggtaaactggtccgtctgagcggtcaagattctcgtcgtggtactttcagccagcgtcactctgtgctgatcgatcgtcacacgggtgaagaattcaccccgctgcaactgctggcgaccaactccgatggctctcctaccggtggtaaattcctggtatacgactctccactgtctgaatatgctgcagttggcttcgaatacggttacactgttggtaacccggacgctgttgtgctgtgggaagctcagttcggcgacttcgtaaatggcgcgcagtccatcattgacgaattcatttcctctggcgaagcgaaatggggccagctgtccaacgtcgtgctgctgctgccacacggccatgaaggtcagggtccggatcatacttctgcgcgcatcgagcgtttcctgcagctgtgggccgagggctccatgaccatcgccatgccgtccaccccgtctaattattttcacctgctgcgccgtcacgcgctggacggtatccagcgcccgctgattgttttcaccccgaaatccatgctgcgccacaaagcggcagtcagcgagattaaagatttcaccgaaatcaaattccgctccgtcctggaagaaccgacctatgaagacggcatcggtgaccgcaacaaggtaagccgcattctgctgacctccggcaaactgtattacgagctggcagctcgcaaggcgaaggataaccgcaacgatctggcaatcgtgcgcctggaacagctggcgccgctgccgcgtcgccgtctgcgtgaaaccctggatcgctatgaaaacgtaaaagagttcttctgggttcaagaagagccggcaaaccagggcgcttggccgcgttttggcctggagctgccggagctgctgccggacaagctggccggtatcaaacgtatctcccgtcgtgctatgagcgccccttctagcggttcttctaaagttcatgctgttgaacagcaagaaatcctggacgaagcgttcggctaa

SEQ ID NO: 22 - protein, 371 aa, Porphyromonas gingivalis
MQLFKLKSVTHHFDTFAEFAKEFCLGERDLVITNEFIYEPYMKACQLPCHFVMQEKYGQGEPSDEMMNNILADIRNIQFDRVIGIGGGTVIDISKLFVLKGLNDVLDAFDRKIPLIKEKELIIVPTTCGTGSEVTNISIAEIKSRHTKMGLADDAIVADHAIIIPELLKSLPFHFYACSAIDALIHAIESYVSPKASPYSRLFSEAAWDIILEVFKKIAEHGPEYRFEKLGEMIMASNYAGIAFGNAGVGAVHALSYPLGGNYHVPHGEANYQFFTEVFKVYQKKNPFGYIVELNWKLSKILNCQPEYVYPKLDELLGCLLTKKPLHEYGMKDEEVRGFAESVLKTQQRLLANNYVELTVDEIEGIYRRLY

SEQ ID NO: 23 - DNA, 1116 bp, Porphyromonas gingivalis
atgcaactgttcaaactgaaatcagtcacacatcacttcgatactttcgcggaatttgccaaagagttctgtcttggagaacgtgatttagtaattaccaacgaattcatttacgaaccgtatatgaaggcatgtcagttgccctgccattttgttatgcaggagaaatatgggcaaggcgagccatctgacgagatgatgaataacatcttggcagacatccgtaatatccagtttgaccgcgtgatcggtattgggggtggtacggttattgacatctcgaaattatttgtgctgaaaggactaaatgatgtgctcgatgcgttcgatcgcaagataccgctgattaaagagaaagaactgatcattgtgcccaccacatgcgggacgggtagcgaggtgacgaatatttcgatcgcggagatcaaaagccgtcataccaaaatgggtttggctgacgatgctattgttgcagaccacgcgatcatcataccagagcttctgaaaagcctgccgttccatttttatgcatgcagtgcaatagatgctctgatccatgccatcgagtcatatgtttctcctaaagccagtccatattctcgtctgttcagtgaggcggcatgggatattatcctggaggtattcaagaaaatagccgaacacggccctgaataccgctttgagaagctgggagaaatgatcatggcctccaactatgctggtatagccttcgggaatgcaggcgtgggtgccgttcacgctctaagctatccattgggaggcaattatcatgtgccgcatggcgaggctaactatcagttttttacagaggtctttaaagtataccaaaagaaaaatcctttcggctatatagtcgaactcaactggaagctgtccaagattctgaactgtcagcctgaatacgtctatccgaaactggatgagttactcggctgtcttctgaccaaaaaaccgctgcacgaatacggcatgaaagatgaagaggtacgtggatttgcggaatcagtgcttaagactcagcagcggttgctcgcgaataattatgttgagcttactgttgatgaaattgaaggtatctacagacgactgtactaa

SEQ ID NO: 24 - DNA, 1286 bp, Escherichia coli
tttaaccgttcagttgaaggttgcgcctacactaagcatagttgttgatgaatttttcaatatcgccatagctttcaattatatttgaaattttgtaaaatatttttagtagcttaaatgtgattcaacatcactggagaaagtcttatgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgacgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactaccatctgttgaacgaagccgccttcgatcagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtaattcaggatgacgtattccgtcgcctgtctgcctgccacaacgtgctatttaccgggcaccaggcattcctgacagcagaagctctgaccagtatttctcagactacgctgcaaaacttaagcaatctggaaaaaggcgaaacctgcccgaacgaactggtttaatcttgccgctcccctgcattccaggggagctgattcagataatccccaatgacctttcatcctctattcttaaaatagccctgagtcagaaactgtaattgagaaccacaatgaagaaagtagccgcgctcgttgcgctaagcctgctg

SEQ ID NO: 25 - DNA, 2967 bp, Escherichia coli
aagcgttcattatggtgctgccggtcgcgatgtttgttgccagcggttttgagcacagtatcgcaaacatgtttatgatcccgatgggtattgtaatccgcgacttcgcatctccggaattctggaccgctgtcggttctgcaccggaaaatttttctcacctgaccgtgatgaacttcatcactgataacctgattccggttacgatcggtaatattatcggcggtggtttgttggttgggttgacatactgggtcatttacctgcgtgaaaacgatcaccattaatggttgtcgaagtacgcagtaaataaaaaatccacttaagaaggtaggtgttacatgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaaagcgttggaaaaagttgttggtctacagactgaagctccgctgaaacgtgctcttatcccgttcggtggtatcaaaatgatcgagggttcctgcaaagcgtacaaccgcgaactggacccgatgatcaaaaaaatcttcactgaataccgtaaaactcacaaccagggcgtgttcgacgtttacactccggacatcctgcgttgccgtaaatccggtgttctgaccggtctgccagatgcttatggccgtggccgtatcatcggtgactaccgtcgcgttgcgctgtacggtatcgactacctgatgaaagacaaatacgctcagttcacctctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcaatatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaactggtaacaccccagacggtcgtcgtgctggcgcgccgttcggaccgggtgctaacccgatgcacggtcgtgaccagaaaggtgctgtagcgtctctgacttccgttgctaaactaccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaattagatttgactgaaatcgtacagtaaaaagcgtacaataaaggctccacgaaagtggggccttttttagcacgagagccttttttgtcagctatctatactttaaggtgactgccaaaacagactcgacgtagccttcgagctgcgcaccaacacggcctcagatgggccacatctggagaaacaccgcaatgtcagttattggtcgcattcactcctttgaatcctgtggaaccgtagacggcccaggtattcgctttatcacctttttccagggctgcctgatgcgctgcctgtattgtcataaccgcgacacctgggatacgcatggcggtaaagaagt

SEQ ID NO: 26 - DNA, 3478 bp, Escherichia coli
gacagcatttttcacctcctaactacttaaaattgctatcattcgttattgttatctagttgtgcaaaacatgctaatgtagccaccaaatcatactacaatttattaactgttagctataatggcgaaaagcgatgctgaaaggtgtcagctttgcaaaaatttgatttggatcacgtaatcagtacccagaagtgagtaatcttgcttacgccacctggaagtgacgcattagagataataactctaatgtttaaactcttttagtaaatcacagtgagtgtgagcgcgagtaagcttttgattttcataggttaagcaaatcatcaccgcactgactatactctcgtattcgagcagatgatttactaaaaaagtttaacattatcaggagagcattatggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggctcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcgttcgatgaccagtgcaccggcgctaacccgcgttacccgctgatctccgagctgaaacagatcctgctggatacctactacggtcgtgattatgtagaaggtgaaactgcagcgaaaaaagaagccgctccggctaaagctgagaaaaaagcgaaaaaatccgcttaatcagtagcgctgtctggcaatataaacggccccttctggggccgtttttttgtttacccaaagcaacttttccataaaccgacagcattagccttcatcatatttgcgacgatgtataacgcctaaacacagggatattgtactttacaggtcacaagtcaacgtcggtgcttaagagccctgtgaggcgtatagcggcgttaaaaaactgccgagaagggtatatagcccggaagaagtgcgtaaaacgaactgacaggataaaagtgccctgctcaccctgtcagtaaagaaattcttattaatcgtggcgatgcctttcctgaatagccgttaatgagccgacttgtaacgcctctatatagtgtttacggcatacagaaacgtatcgttcattaccac

SEQ ID NO: 27 - protein, 312 aa, Escherichia coli
MKVAVLGAAGGIGQALALLLKTQLPSGSELSLYDIAPVTPGVAVDLSHIPTAVKIKGFSGEDATPALEGADVVLISAGVARKPGMDRSDLFNVNAGIVKNLVQQVSKTCPKACIGIITNPVNTTVAIAAEVLKKAGVYDKNKLFGVTTLDIIRSNTFVAELKGKQPGEVEVPVIGGHSGVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQSALEGMLDTLKKDIALGEEFVNK

SEQ ID NO: 28 - DNA, 939 bp, Escherichia coli
atgaaagtcgcagtcctcggcgctgctggcggtattggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggtgtagcgcgtaaaccgggtatggatcgttccgacctgtttaacgttaacgccggcatcgtgaaaaacctggtacagcaagtttcgaaaacctgcccgaaagcgtgcattggtattatcactaacccggttaacaccacagttgcgattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctggtgttaccattctgccgctgctgtcacaggttcctggcgttagttttaccgagcaggaagtggctgatctgaccaaacgtatccagaacgcaggtactgaagtggttgaagcgaaagccggtggcgggtctgcaaccctgtctatgggccaggcagctgcacgttttggtctgtctctggtacgcgcactgcagggcgaacaaggcgttgtcgaatgtgcctatgttgaaggcgacggtcagtacgcacgtttcttctctcaaccgctgctgctgggtaaaaacggcgtggaagagcgtaaatctatcggtaccctgagcgcatttgaacagagcgcactggaaggtatgctggatacgctgaagaaagatatcgccctgggcgaagagttcgttaataagtaa

SEQ ID NO: 29 - protein, 238 aa, Escherichia coli
MQTPHILIVEDELVTRNTLKSIFEAEGYDVFEATDGAEMHQILSEYDINLVIMDINLPGKNGLLLARELREQANVALMFLTGRDNEVDKILGLEIGADDYITKPFNPRELTIRARNLLSRTMNLGTVSEERRSVESYKFNGWELDINSRSLIGPDGEQYKLPRSEFRAMLHFCENPGKIQSRAELLKKMTGRELKPHDRTVDVTIRRIRKHFESTPDTPEIIATIHGEGYRFCGDLED

SEQ ID NO: 30 - DNA, 717 bp, Escherichia coli
atgcagaccccgcacattcttatcgttgaagacgagttggtaacacgcaacacgttgaaaagtattttcgaagcggaaggctatgatgttttcgaagcgacagatggcgcggaaatgcatcagatcctctctgaatatgacatcaacctggtgatcatggatatcaatctgccgggtaagaacggtcttctgttagcgcgtgaactgcgcgagcaggcgaatgttgcgttgatgttcctgactggccgtgacaacgaagtcgataaaattctcggcctcgaaatcggtgcagatgactacatcaccaaaccgttcaacccgcgtgaactgacgattcgtgcacgcaacctgctgtcccgtaccatgaatctgggtactgtcagcgaagaacgtcgtagcgttgaaagctacaagttcaatggttgggaactggatatcaacagccgttcgttgatcggccctgatggcgagcagtacaagctgccgcgcagcgagttccgcgccatgcttcacttctgtgaaaacccaggcaaaattcagtctcgtgctgaactgctgaagaaaatgaccggccgtgagctgaaaccacacgaccgtactgtagacgtgacgatccgccgtattcgtaaacatttcgaatctacgccggatacgccggaaatcatcgccaccatccacggtgaaggttatcgcttctgtggtgatctggaagattaa

SEQ ID NO: 31 - protein, 474 aa, Escherichia coli [sequence omitted]

SEQ ID NO: 32 - DNA, 1425 bp, Escherichia coli
atgagtactgaaatcaaaactcaggtcgtggtacttggggcaggccccgcaggttactccgctgccttccgttgcgctgatttaggtctggaaaccgtaatcgtagaacgttacaacacccttggcggtgtttgcctgaacgtcggctgtatcccttctaaagcactgctgcacgtagcaaaagttatcgaagaagccaaagcgctggctgaacacggtatcgtcttcggcgaaccgaaaaccgatattgacaagattcgtacctggaaagagaaagtgatcaatcagctgaccggtggtctggctggtatggcgaaaggccgcaaagtcaaagtggtcaacggtctgggtaaatttaccggggctaacaccctggaagttgaaggtgagaacggtaaaaccgtgatcaacttcgacaacgcgatcattgcagcgggttctcgcccgatccaactgccgtttattccgcatgaagatccgcgtatctgggactccactgacgcgctggaactgaaagaagtaccagaacgcctgctggtaatgggtggcggtatcatcggtctggaaatgggcaccgtataccacgcgctgggttcacagattgacgtggttgaaatgttcgaccaggttatcccggcagctgacaaagacatcgttaaagtcttcaccaagcgtatcagcaagaaattcaacctgatgctggaaaccaaagttaccgccgttgaagcgaaagaagacggtatttatgtgacgatggaaggcaaaaaagcacccgctgaaccgcagcgttacgacgccgtgctggtagcgattggtcgtgtgccgaacggtaaaaacctcgacgcaggcaaagctggcgtggaagtggacgaccgtggtttcatccgcgttgacaaacagctgcgtaccaacgtaccgcacatctttgctatcggcgatatcgtcggtcagccgatgctggcacacaaaggtgttcacgaaggtcacgttgccgctgaagttatcgccggtaagaaacactacttcgatccgaaagttatcccgtccatcgcctataccgaaccagaagttgcatgggtaggtctgactgagaaagaagcgaaagagaaaggcatcagctatgaaaccgccaccttcccgtgggctgcttctggtcgtgctatcgcttccgactgcgcagacggtatgaccaagctgattttcgacaaagaatctcaccgtgtgatcggtggtgcgattgtcggtaccaacggcggcgagctgctgggtgaaatcggcctggcaatcgaaatgggttgtgatgctgaagacatcgcactgaccatccacgcgcacccgactctgcacgagtctgtgggcctggcggcagaagtgttcgaaggtagcattaccgacctgccgaacccgaaagcgaagaagaagtaa

SEQ ID NO: 33 - protein, 474 aa, Klebsiella pneumoniae
MSTEIKTQVVVLGAGPAGYSAAFRCADLGLETVIVERYSTLGGVCLNVGCIPSKALLHVAKVIEEAKALAEHGIVFGEPKTDIDKIRTWKEKVITQLTGGLAGMAKGRKVKVVNGLGKFTGANTLEVEGENGKTVINFDNAIIAAGSRPIQLPFIPHEDPRVWDSTDALELKSVPKRMLVMGGGIIGLEMGTVYHALGSEIDVVEMFDQVIPAADKDVVKVFTKRISKKFNLMLETKVTAVEAKEDGIYVSMEGKKAPAEAQRYDAVLVAIGRVPNGKNLDAGKAGVEVDDRGFIRVDKQMRTNVPHIFAIGDIVGQPMLAHKGVHEGHVAAEVISGLKHYFDPKVIPSIAYTEPEVAWVGLTEKEAKEKGISYETATFPWAASGRAIASDCADGMTKLIFDKETHRVIGGAIVGTNGGELLGEIGLAIEMGCDAEDIALTIHAHPTLHESVGLAAEVFEGSITDLPNAKAKKK

SEQ ID NO: 34 - DNA, 1425 bp, Klebsiella pneumoniae
atgagtactgaaatcaaaactcaggtcgtggtacttggggcaggccccgcaggttactctgcagccttccgttgcgctgatttaggtctggaaaccgtcatcgtagaacgttacagcaccctcggtggtgtttgtctgaacgtgggttgtatcccttctaaagcgctgctgcacgtggcaaaagttatcgaagaagcgaaagcgctggccgaacacggcatcgttttcggcgaaccgaaaactgacattgacaagatccgcacctggaaagaaaaagtcatcactcagctgaccggtggtctggctggcatggccaaaggtcgtaaagtgaaggtggttaacggtctgggtaaatttaccggcgctaacaccctggaagtggaaggcgaaaacggcaaaaccgtgatcaacttcgacaacgccatcatcgcggcgggttcccgtccgattcagctgccgtttatcccgcatgaagatccgcgcgtatgggactccaccgacgcgctggaactgaaatctgtaccgaaacgcatgctggtgatgggcggcggtatcatcggtctggaaatgggtaccgtataccatgcgctgggttcagagattgacgtggtggaaatgttcgaccaggttatcccggctgccgacaaagacgtggtgaaagtcttcaccaaacgcatcagcaagaaatttaacctgatgctggaaaccaaagtgactgccgttgaagcgaaagaagacggtatttacgtttccatggaaggtaaaaaagcaccggcggaagcgcagcgttacgacgcagtgctggtcgctatcggccgcgtaccgaatggtaaaaacctcgatgcaggtaaagctggcgtggaagttgacgatcgcggcttcatccgcgttgacaaacaaatgcgcaccaacgtgccgcacatctttgctatcggcgatatcgtcggtcagccgatgctggcgcacaaaggtgtccatgaaggccacgttgccgcagaagttatctccggtctgaaacactacttcgatccgaaagtgatcccatccatcgcctacactgaaccagaagtggcatgggtcggtctgaccgagaaagaagcgaaagagaaaggcatcagctacgaaaccgccaccttcccgtgggctgcttccggccgtgctatcgcttctgactgcgcagatggtatgaccaaactgatcttcgacaaagagacccaccgtgttatcggcggcgcgattgtcggcaccaacggcggcgagctgctgggtgagatcggcctggctatcgagatgggctgtgacgctgaagacatcgccctgaccatccacgctcacccgactctgcacgagtccgttggcctggcggcggaagtgttcgaaggcagcatcaccgacctgccaaacgccaaagcgaagaaaaagtaa

SEQ ID NO: 35 - protein, 474 aa, Artificial Sequence
  Synthetic (mutant of Klebsiella pneumoniae LpdA)
MSTEIKTQVVVLGAGPAGYSAAFRCADLGLETVIVERYSTLGGVCLNVGCIPSKALLHVAKVIEEAKALAEHGIVFGEPKTDIDKIRTWKEKVITQLTGGLAGMAKGRKVKVVNGLGKFTGANTLEVEGENGKTVINFDNAIIAAGSRPIQLPFIPHEDPRVWDSTDALELKSVPKRMLVMGGGIIGLEMGTVYHALGSEIDVVEMFDQVIPAADKDVVKVFTKRISKKFNLMLETKVTAVEAKEDGIYVSMEGKKAPAEAQRYDAVLVAIGRVPNGKNLDAGKAGVEVDDRGFIRVDKQMRTNVPHIFAIGDIVGQPMLAHKGVHEGHVAAEVISGLKHYFDPKVIPSIAYTKPEVAWVGLTEKEAKEKGISYETATFPWAASGRAIASDCADGMTKLIFDKETHRVIGGAIVGTNGGELLGEIGLAIEMGCDAEDIALTIHAHPTLHESVGLAAEVFEGSITDLPNAKAKKK

SEQ ID NO: 36 - DNA, 1425 bp, Artificial Sequence
  Synthetic (mutant of Klebsiella pneumoniae lpdA)
atgagtactgaaatcaaaactcaggtcgtggtacttggggcaggccccgcaggttactctgcagccttccgttgcgctgatttaggtctggaaaccgtcatcgtagaacgttacagcaccctcggtggtgtttgtctgaacgtgggttgtatcccttctaaagcgctgctgcacgtggcaaaagttatcgaagaagcgaaagcgctggccgaacacggcatcgttttcggcgaaccgaaaactgacattgacaagatccgcacctggaaagaaaaagtcatcactcagctgaccggtggtctggctggcatggccaaaggtcgtaaagtgaaggtggttaacggtctgggtaaatttaccggcgctaacaccctggaagtggaaggcgaaaacggcaaaaccgtgatcaacttcgacaacgccatcatcgcggcgggttcccgtccgattcagctgccgtttatcccgcatgaagatccgcgcgtatgggactccaccgacgcgctggaactgaaatctgtaccgaaacgcatgctggtgatgggcggcggtatcatcggtctggaaatgggtaccgtataccatgcgctgggttcagagattgacgtggtggaaatgttcgaccaggttatcccggctgccgacaaagacgtggtgaaagtcttcaccaaacgcatcagcaagaaatttaacctgatgctggaaaccaaagtgactgccgttgaagcgaaagaagacggtatttacgtttccatggaaggtaaaaaagcaccggcggaagcgcagcgttacgacgcagtgctggtcgctatcggccgcgtaccgaatggtaaaaacctcgatgcaggtaaagctggcgtggaagttgacgatcgcggcttcatccgcgttgacaaacaaatgcgcaccaacgtgccgcacatctttgctatcggcgatatcgtcggtcagccgatgctggcgcacaaaggtgtccatgaaggccacgttgccgcagaagttatctccggtctgaaacactacttcgatccgaaagtgatcccatccatcgcctacactaagccagaagtggcatgggtcggtctgaccgagaaagaagcgaaagagaaaggcatcagctacgaaaccgccaccttcccgtgggctgcttccggccgtgctatcgcttctgactgcgcagatggtatgaccaaactgatcttcgacaaagagacccaccgtgttatcggcggcgcgattgtcggcaccaacggcggcgagctgctgggtgagatcggcctggctatcgagatgggctgtgacgctgaagacatcgccctgaccatccacgctcacccgactctgcacgagtccgttggcctggcggcggaagtgttcgaaggcagcatcaccgacctgccaaacgccaaagcgaagaaaaagtaa

SEQ ID NO: 37 - protein, 427 aa, Escherichia coli
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFRLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 38 - DNA, 1284 bp, Escherichia coli
atggctgatacaaaagcaaaactcaccctcaacggggacacagctgttgaactggatgtgctgaaaggcacgctgggtcaagatgttattgatatccgtactctcggttcaaaaggtgtgttcacctttgacccaggcttcacttcaaccgcatcctgcgaatctaaaattacttttattgatggtgatgaaggtattttgctgcaccgcggtttcccgatcgatcagctggcgaccgattctaactacctggaagtttgttacatcctgctgaatggtgaaaaaccgactcaggaacagtatgacgaatttaaaactacggtgacccgtcataccatgatccacgagcagattacccgtctgttccacgctttccgtcgcgactcacatccaatggcagtcatgtgtggtattaccggcgcgctggcggcgttctatcacgactcgctggatgttaacaatcctcgtcatcgtgaaattgccgcgttccgcctgctgtcgaaaatgccgaccatggccgcgatgtgttacaagtattccattggtcagccatttgtttatccgcgcaacgatctctcctatgccggtaacttcctgaatatgatgttctccacgccgtgcgaaccgtatgaagttaatccgattctggaacgtgctatggaccgtattctgatcctgcacgctgaccatgaacagaacgcctctacctccaccgtgcgtaccgctggctcttcgggtgcgaacccgtttgcctgtatcgcagcaggtattgcttcactgtggggacctgcgcacggtggtgctaacgaagcggcgctgaaaatgctggaagaaattagctccgttaaacacattccggaatttgttcgtcgtgcgaaagataaaaatgattctttccgcctgatgggcttcggtcaccgcgtgtacaaaaattacgacccgcgcgccaccgtaatgcgtgaaacctgccatgaagttctgaaagagctgggcaccaaagatgacctgctggaagtggctatggagctggaaaacatcgcgctgaacgacccgtactttatcgagaagaaactgtacccgaacgtcgatttctactctggtatcatcctgaaagcgatgggtattccgtcttccatgttcaccgtcattttcgcaatggcacgtaccgttggctggatcgcccactggagcgaaatgcacagtgacggtatgaagattgcccgtccgcgtcagctgtatacaggatatgaaaaacgcgactttaaaagcgatatcaagcgttaa

SEQ ID NO: 39 - protein, 427 aa, Artificial Sequence
  Synthetic (mutant of GltA)
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFLLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 40 - DNA, 1284 bp, Artificial Sequence
  Synthetic (mutant of gltA)
atggctgatacaaaagcaaaactcaccctcaacggggacacagctgttgaactggatgtgctgaaaggcacgctgggtcaagatgttattgatatccgtactctcggttcaaaaggtgtgttcacctttgacccaggcttcacttcaaccgcatcctgcgaatctaaaattacttttattgatggtgatgaaggtattttgctgcaccgcggtttcccgatcgatcagctggcgaccgattctaactacctggaagtttgttacatcctgctgaatggtgaaaaaccgactcaggaacagtatgacgaatttaaaactacggtgacccgtcataccatgatccacgagcagattacccgtctgttccacgctttccgtcgcgactcacatccaatggcagtcatgtgtggtattaccggcgcgctggcggcgttctatcacgactcgctggatgttaacaatcctcgtcatcgtgaaattgccgcgttcctcctgctgtcgaaaatgccgaccatggccgcgatgtgttacaagtattccattggtcagccatttgtttatccgcgcaacgatctctcctatgccggtaacttcctgaatatgatgttctccacgccgtgcgaaccgtatgaagttaatccgattctggaacgtgctatggaccgtattctgatcctgcacgctgaccatgaacagaacgcctctacctccaccgtgcgtaccgctggctcttcgggtgcgaacccgtttgcctgtatcgcagcaggtattgcttcactgtggggacctgcgcacggtggtgctaacgaagcggcgctgaaaatgctggaagaaattagctccgttaaacacattccggaatttgttcgtcgtgcgaaagataaaaatgattctttccgcctgatgggcttcggtcaccgcgtgtacaaaaattacgacccgcgcgccaccgtaatgcgtgaaacctgccatgaagttctgaaagagctgggcaccaaagatgacctgctggaagtggctatggagctggaaaacatcgcgctgaacgacccgtactttatcgagaagaaactgtacccgaacgtcgatttctactctggtatcatcctgaaagcgatgggtattccgtcttccatgttcaccgtcattttcgcaatggcacgtaccgttggctggatcgcccactggagcgaaatgcacagtgacggtatgaagattgcccgtccgcgtcagctgtatacaggatatgaaaaacgcgactttaaaagcgatatcaagcgttaa

SEQ ID NO: 41 - DNA, 71 bp, Artificial Sequence
  Synthetic (forward primer)
atgaaactcgccgtttatagcacaaaacagtacgacaagaagtacctgcataggtgacactatagaacgcg

SEQ ID NO: 42 - DNA, 70 bp, Artificial Sequence
  Synthetic (reverse primer)
ttaaaccagttcgttcgggcaggtttcgcctttttccagattgcttaagttagtggatctgatgggtacc

SEQ ID NO: 43 - DNA, 71 bp, Artificial Sequence
  Synthetic (forward primer)
atgtccgagcttaatgaaaagttagccacagcctgggaaggttttaccaataggtgacactatagaacgcg

SEQ ID NO: 44 - DNA, 70 bp, Artificial Sequence
  Synthetic (reverse primer)
ttacatagattgagtgaaggtacgagtaataacgtcctgctgctgttctttagtggatctgatgggtacc

SEQ ID NO: 45 - DNA, 71 bp, Artificial Sequence
  Synthetic (forward primer)
atggctgttactaatgtcgctgaacttaacgcactcgtagagcgtgtaaataggtgacactatagaacgcg

SEQ ID NO: 46 - DNA, 70 bp, Artificial Sequence
  Synthetic (reverse primer)
ttaagcggattttttcgcttttttctcagctttagccggagcggcttctttagtggatctgatgggtacc

SEQ ID NO: 47 - DNA, 71 bp, Artificial Sequence
  Synthetic (forward primer)
atgaaagtcgcagtcctcggcgctgctggcggtattggccaggcgcttgctaggtgacactatagaacgcg

SEQ ID NO: 48 - DNA, 70 bp, Artificial Sequence
  Synthetic (reverse primer)
ttacttattaacgaactcttcgcccagggcgatatctttcttcagcgtattagtggatctgatgggtacc

SEQ ID NO: 49 - DNA, 71 bp, Artificial Sequence
  Synthetic (forward primer)
atgcagaccccgcacattcttatcgttgaagacgagttggtaacacgcaataggtgacactatagaacgcg

SEQ ID NO: 50 - DNA, 70 bp, Artificial Sequence
  Synthetic (reverse primer)
ttaatcttccagatcaccacagaagcgataaccttcaccgtggatggtggtagtggatctgatgggtacc

SEQ ID NO: 51 - DNA, 20 bp, Artificial Sequence
  Synthetic (forward primer)
tacactaagcatagttgttg

SEQ ID NO: 52 - DNA, 20 bp, Artificial Sequence
  Synthetic (reverse primer)
ctttcttcattgtggttctc

SEQ ID NO: 53 - DNA, 20 bp, Artificial Sequence
  Synthetic (forward primer)
gggtcatttacctgcgtgaa

SEQ ID NO: 54 - DNA, 20 bp, Artificial Sequence
  Synthetic (reverse primer)
agtctgttttggcagtcacc

SEQ ID NO: 55 - DNA, 20 bp, Artificial Sequence
  Synthetic (forward primer)
caccgcactgactatactct

SEQ ID NO: 56 - DNA, 20 bp, Artificial Sequence
  Synthetic (reverse primer)
gatgaaggctaatgctgtcg

SEQ ID NO: 57 - DNA, 20 bp, Artificial Sequence
  Synthetic (forward primer)
ggttcctgattacggcaatt

SEQ ID NO: 58 - DNA, 20 bp, Artificial Sequence
  Synthetic (reverse primer)
attcaggaatatccggcaac

SEQ ID NO: 59 - DNA, 20 bp, Artificial Sequence
  Synthetic (forward primer)
ttgacgttgatggaaagtgc

SEQ ID NO: 60 - DNA, 20 bp, Artificial Sequence
  Synthetic (reverse primer)
ccgaaaatgaaagccagtaa

SEQ ID NO: 61 - DNA, 32 bp, Artificial Sequence
  Synthetic (forward primer)
aggaaacagaccatgattaaagacacgctagt

SEQ ID NO: 62 - DNA, 37 bp, Artificial Sequence
  Synthetic (reverse primer)
catctgtttcgaattcttaaccggcgagtacacatct

SEQ ID NO: 63 - DNA, 28 bp, Artificial Sequence
  Synthetic (reverse primer)
ggaaacagaattcatggaaataaaagag

SEQ ID NO: 64 - DNA, 29 bp, Artificial Sequence
  Synthetic (reverse primer)
cctgtgtgattagagttcccagatctctt

SEQ ID NO: 65 - DNA, 31 bp, Artificial Sequence
  Synthetic (forward primer)
cagaattcatgcaactgttcaaactgaaatc

SEQ ID NO: 66 - DNA, 31 bp, Artificial Sequence
  Synthetic (reverse primer)
atccccggttagtacagtcgtctgtagatac

SEQ ID NO: 67 - DNA, 31 bp, Artificial Sequence
  Synthetic (forward primer)
aggaaacagaattcatgtaccgtaaattccg

SEQ ID NO: 68 - DNA, 30 bp, Artificial Sequence
  Synthetic (reverse primer)
caggtcgactctagttagccgaacgcttcg

SEQ ID NO: 69 - DNA, 28 bp, Artificial Sequence
  Synthetic (forward primer)
ccatcgcctacactaagccagaagtggc

SEQ ID NO: 70 - DNA, 28 bp, Artificial Sequence
  Synthetic (reverse primer)
gccacttctggcttagtgtaggcgatgg

SEQ ID NO: 71 - DNA, 82 bp, Artificial Sequence
  Synthetic (forward primer)
gccgctgcggcctgaaagacgacgggtatgaccgccggagataaatatatagaggtcatgaactgtctgcttacataaacag

SEQ ID NO: 72 - DNA, 77 bp, Artificial Sequence
  Synthetic (reverse primer)
taaaaaaagcggcgtggttagccgcttttttaattgccggatgttccggcaaacgaacaattggtcggtcatttcgc

SEQ ID NO: 73 - DNA, 90 bp, Artificial Sequence
  Synthetic (forward primer)
ccggatccgccgctgcggcctgaaagacgacgggtatgaccgccggagataaatatatagaggtcatgatgagtactgaaatcaaaactc

SEQ ID NO: 74 - DNA, 89 bp, Artificial Sequence
  Synthetic (reverse primer)
gggtcgactaaaaaaagcggcgtggttagccgcttttttaattgccggatgttccggcaaacgaacaattactttttcttcgctttggc

SEQ ID NO: 75 - DNA, 19 bp, Artificial Sequence
  Synthetic (forward primer)
catcattaacaacacgctg

SEQ ID NO: 76 - DNA, 19 bp, Artificial Sequence
  Synthetic (reverse primer)
cgacagtaaccatactgtc

SEQ ID NO: 77 - DNA, 19 bp, Artificial Sequence
  Synthetic (forward primer)
aattgccgcgttcctcctg

SEQ ID NO: 78 - DNA, 17 bp, Artificial Sequence
  Synthetic (reverse primer)
tcgacagcaggaggaac

SEQ ID NO: 79 - DNA, 80 bp, Artificial Sequence
  Synthetic (forward primer)
gtgcgaaggcaaatttaagttccggcagtcttacgtaataaggcgctaaggagaccttaactgtctgcttacataaacag

SEQ ID NO: 80 - DNA, 78 bp, Artificial Sequence
  Synthetic (reverse primer)
ataaaaattaacccgccatttgaacggcgggttaaaatatttacaacttagcaatcaaccattggtcggtcatttcgc

SEQ ID NO: 81 - DNA, 67 bp, Artificial Sequence
  Synthetic (forward primer)
gtgcgaaggcaaatttaagttccggcagtcttacgtaataaggcgctaaggagaccttaaatggctg

SEQ ID NO: 82 - DNA, 78 bp, Artificial Sequence
  Synthetic (reverse primer)
ataaaaattaacccgccatttgaacggcgggttaaaatatttacaacttagcaatcaaccattaacgcttgatatcgc

SEQ ID NO: 83 - DNA, 21 bp, Artificial Sequence
  Synthetic (forward primer)
ggacagttattagtggtagac

SEQ ID NO: 84 - DNA, 22 bp, Artificial Sequence
  Synthetic (reverse primer)
gatgtatttcacacggtgcttc

(19) United States
(12) Patent Application Publication
(10) Pub. No.: US 2015/0024447 A1
(43) Pub. Date: Jan. 22, 2015

(54) BUTYRALDEHYDE DEHYDROGENASE MUTANT, POLYNUCLEOTIDE ENCODING THE MUTANT, VECTOR AND MICROORGANISM HAVING THE POLYNUCLEOTIDE, AND METHOD OF PRODUCING 1,4-BUTANEDIOL USING THE SAME

(71) Applicant: Samsung Electronics Co., Ltd., Suwon-si (KR)

(72) Inventors: Ji-eun KIM, Seoul (KR); Jin-woo Park, Daejeon (KR); Jin-hwan Park, Suwon-si (KR); Yu-kyung Jung, Hwaseong-si (KR); Hwa-young Cho, Hwaseong-si (KR); Jae Chan Park, Yongin-si (KR); Kwang-myung Cho, Seongnam-si (KR)

(21) Appl. No.: 14/335,632

(22) Filed: Jul. 18, 2014

(30) Foreign Application Priority Data
Jul. 19, 2013 (KR) 10-2013-0085691

(51) Int. Cl. C12P 7/18; C12N 9/02

(52) U.S. Cl. CPC C12P 7/18; C12N 9/0008; C12Y 102/01057
USPC 435/158; 435/190; 435/252.33

(57) ABSTRACT

A mutant butyraldehyde dehydrogenase (Bld), a polynucleotide having a nucleotide encoding the mutant, a vector including the polynucleotide, a microorganism including a nucleotide encoding the mutant, and a method of producing 1,4-butanediol using the same.

### CROSS-REFERENCE TO RELATED APPLICATIONS

This application claims the benefit of Korean Patent Application No. 10-2013-0085691, filed on Jul. 19, 2013, in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 133,850 Byte ASCII (Text) file named “715755_ST25.TXT,” created on Jul. 16, 2014.

### BACKGROUND

1. Field

The present disclosure relates to an enzyme used for synthesis of 1,4-butanediol, a microorganism producing the 1,4-butanediol, and a method of producing 1,4-butanediol using the same.

2. Description of the Related Art

Bio-plastic is a polymer synthesized from raw material biomass which is a regenerative plant-derived resource capable of replacing conventional fossil fuel. Since biomass consumes carbon dioxide in the air in the photosynthesis process, bio-plastic is a very useful material in view of reducing carbon emissions. Bio-plastic may be extensively utilized in low-carbon green growth industries because bio-plastic may replace fossil fuels and reduce carbon dioxide without causing an environmental pollution problem.

Biodegradable polymer substances are suggested as an alternative to synthetic polymer materials. 1,4-butanediol (1,4-BDO) is a solvent produced worldwide in amounts of 1.3 million tons each year from petroleum-based materials such as acetylene, butane, propylene, and butadiene.

The 1,4-butanediol is important as it is used throughout the entire chemical industry for the production of various chemicals such as polymers, solvents, and fine chemistry intermediates. Most of the chemicals having a carbon number of four are currently synthesized from 1,4-butanediol or maleic anhydride, but the chemical production process needs to be improved or replaced by a newly developed process as production costs are increasing due to rising oil prices. Alternative processes are required to effectively produce a commercial quantity of 1,4-butanediol and precursors thereof. Biological processes using microorganisms are suggested as the alternative processes.

A microorganism capable of effectively producing 1,4-butanediol was developed by using a butyraldehyde dehydrogenase mutant.

### SUMMARY

An aspect of the present invention provides a butyraldehyde dehydrogenase (Bid) having a catalytic activity of converting 4-hydroxybutyryl CoA (4HB-CoA) to 4-hydroxybutyraldehyde (4HB aldehyde), wherein at least one amino acid residue is mutated.

Another aspect of the present invention provides a polynucleotide having a nucleotide sequence encoding the butyraldehyde dehydrogenase (Bid).

Another aspect of the present invention provides a vector including the polynucleotide having the nucleotide sequence encoding the butyraldehyde dehydrogenase (Bld).

Another aspect of the present invention provides a microorganism including a gene encoding the butyraldehyde dehydrogenase.

Another aspect of the present invention provides a method of producing 1,4-butanediol using the butyraldehyde dehydrogenase and/or the microorganism expressing the butyraldehyde dehydrogenase.

### BRIEF DESCRIPTION OF THE DRAWINGS

These and/or other aspects will become apparent and more readily appreciated from the following description of the embodiments, taken in conjunction with the accompanying drawings of which:

FIG. 1 is a pMloxC vector map;

FIG. 2 is a pTrc99a vector map;

FIG. 3 is a pTac15ksucD-4hbd-sucA vector map.

FIG. 4 is a graph comparing 1,4-butanediol production of strains expressing a wild-type butyraldehyde dehydrogenase and butyraldehyde dehydrogenase mutants of BldH, BldI, BldJ or BldL, respectively, wherein the Y-axis of FIGS. 4-10 is a percentage of 1,4-butanediol production of the strains relative to 1,4-butanediol production of the control group.

FIG. 5 is a graph comparing 1,4-butanediol production of strains expressing a wild-type butyraldehyde dehydrogenase and a butyraldehyde dehydrogenase mutant of BldS2 wherein the Y-axis of FIGS. 4-10 is a percentage of 1,4-butanediol production of the strains relative to 1,4-butanediol production of the control group.

FIG. 6 is a graph comparing 1,4-butanediol production of strains expressing a butyraldehyde dehydrogenase mutant of BldI or BldS, which is an Escherichia (ATCC 9637) strain to which pTrc99a (bidI) and pTrc99a (cat2) are introduced (Δ IdhA Δ pflB Δ adhE Δ mdh Δ arcA) or an Escherichia (ATCC 9637) strain to which pTrc99a (bldS) and pTrc99a (cat2) are introduced (Δ IdhA Δ pflB Δ adhE Δ mdh Δ arcA) wherein the Y-axis of FIGS. 4-10 is a percentage of 1,4-butanediol production of the strains relative to 1,4-butanediol production of the control group.

FIG. 7 is a graph comparing 1,4-butanediol production of strains expressing a wild-type butyraldehyde dehydrogenase and butyraldehyde dehydrogenase mutants of BldI, BldS or Bld (G226I) wherein the Y-axis of FIGS. 4-10 is a percentage of 1,4-butanediol production of the strains relative to 1,4-butanediol production of the control group.

FIG. 8 is a graph comparing 1,4-butanediol production of strains expressing a wild-type butyraldehyde dehydrogenase (Bld_{wt}) and butyraldehyde dehydrogenase mutants of BldH, BldI, BldJ or BldL wherein the Y-axis of FIGS. 4-10 is a percentage of 1,4-butanediol production of the strains relative to 1,4-butanediol production of the control group.

FIG. 9 is a graph comparing 1,4-butanediol production of strains expressing a butyraldehyde dehydrogenase mutant of BldI or BldS, which is an Escherichia (ATCC 9637) strain to which pTrc99a (bidI) and pTrc99a (cat2) are introduced (Δ IdhA Δ pflB Δ adhE Δ mdh Δ arcA) or an Escherichia (ATCC 9637) strain to which pTrc99a (bldS) and pTrc99a (cat2) are introduced (Δ IdhA Δ pflB Δ adhE Δ mdh Δ arcA) wherein the Y-axis of FIGS. 4-10 is a percentage of 1,4-butanediol production of the strains relative to 1,4-butanediol production of the control group.

FIG. 10 is a graph comparing 1,4-butanediol production of strains expressing a wild-type butyraldehyde dehydrogenase and butyraldehyde dehydrogenase mutants of BldI, BldS or Bld (G226I), wherein the Y-axis of FIGS. 4-10 is a percentage of 1,4-butanediol production of the strains relative to 1,4-butanediol production of the control group.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

An aspect of the present invention provides a butyraldehyde dehydrogenase (Bid) having an amino acid sequence of SEQ ID NO: 1 and a catalytic activity of converting 4-hydroxybutyryl CoA (4HB-CoA) to 4-hydroxybutyraldehyde (4HB aldehyde), wherein at least one amino acid residue at a NADH-binding site is mutated.

Butyraldehyde dehydrogenase has a catalytic activity of converting 4-hydroxybutyryl CoA to 4-hydroxybutyraldehyde by using NADH or NADPH. A binding site of butyraldehyde dehydrogenase with NADH or NADPH may be Met371, Leu273, Met227, Gly226 of SEQ ID NO: 1 or a combination thereof.

In the butyraldehyde dehydrogenase, Met371, Leu273, Met227, Gly226 of SEQ ID NO: 1, or a combination of such positions, may be substituted with other amino acids.

In the butyraldehyde dehydrogenase, Gly226 of SEQ ID NO: 1 may be substituted with another amino acid. This other amino acid may be an amino acid selected from the group consisting of Ile, Leu, Phe, and Tyr. In the butyraldehyde dehydrogenase, Met227 of SEQ ID NO: 1 may be substituted with another amino acid. This other amino acid may be an amino acid selected from the group consisting of Ile, Leu, Gln, and Val. In addition, in butyraldehyde dehydrogenase, Leu273 of SEQ ID NO: 1 may be substituted with Ile. In addition, in the butyraldehyde dehydrogenase, Leu273 of SEQ ID NO: 1 may be substituted with Ile and Met227 of SEQ ID NO: 1 may be substituted with an amino acid selected from the group consisting of Ile, Leu, Gln, and Val.

In addition, a mutant of the butyraldehyde dehydrogenase may be formed by substituting at least one amino acid selected from the group consisting of Asn144, Met227, Ala241, Gly242, Ala243, Gly244, Pro246, Leu273, Pro274, Ile276, Ala277, Lys279, Glu368, His398, Val432, and Thr441 of SEQ ID NO: 1 at the catalytic site with another amino acid. The catalytic site may refer to a site wherein a substrate is bound to a coenzyme. The substrate is 4-hydroxybutyryl CoA, and the coenzyme may be NADH or NADPH. For example, in the butyraldehyde dehydrogenase, a butyraldehyde dehydrogenase mutant may be formed by substituting at the catalytic site Asn144 with Asp, Ala241 with Val, Gly242 with Ser, Ala- with Gly, Gly244 with Ser, Pro246 with Tyr, Leu273 with Ile, Pro274 with Tyr, Ile276 with Leu, Ala277 with Val, Lys279 with Arg, Glu368 with Gln, His398 with Lys, and/or Val432 with Leu, and Thr441 with Asp in SEQ ID NO: 1.

In addition, a mutant of the butyraldehyde dehydrogenase may be formed by substituting at least one amino acid selected from the group consisting of Met91, Ile139, Thr140, Pro141, Ser142, Thr143, Asn166, Gly167, His168, Pro169, Thr203, Met204, Leu207, Asp208, Ile210, Ile211, Lys212, Thr222, Gly223, Gly224, Pro225, Met227, Thr230, Leu231, Ala241, Gly242, Ala243, Gly244, Leu273, Pro274, Cys275, Ser326, Ile327, Asn328, Lys329, Val332, Thr367, Glu368, Leu369, Met370, and Arg396 of SEQ ID NO: 1 with another amino acid.

A mutant of the butyraldehyde dehydrogenase mutant may also be formed by substituting Met91 with Asp, Ile139 with Leu, Thr140 with Lys, Pro141 with Tyr, Ser142 with Gly, Thr143 with Lys, Asn166 with Asp, Gly167 with Ser, His168 with Lys, Pro169 with Tyr, Thr203 with Lys, Met204 with Asp, Leu207 with Ile, Asp208 with Asn, Ile210 with Leu, Ile211 with Leu, Lys212 with Thr, Thr222 with Lys, Gly223 with Ser, Gly224 with Ser, Pro225 with His, Met227 with Lys, Thr230 with Lys, Leu231 with Val, Ala241 with Val, Gly242 with Ser, Ala243 with Val, Gly244 with Ser, Leu273 with Ile, Pro274 with His, Cys275 with Met, Ser326 with Gly, Ile327 with Leu, Asn328 with Asp, Lys329 with Thr, Val332 with Leu, Thr367 with Lys, Glu368 with Gln, Leu369 with Ile, Met370 with Lys, and/or Arg396 with Lys in SEQ ID NO: 1

Another aspect of the present invention provides a butyraldehyde dehydrogenase having the amino acid sequence of SEQ ID NO: 1 wherein 226th, 227th, and 273th amino acid residues or a combination thereof of SEQ ID NO: 1 are mutated. The mutant has a catalytic activity of converting 4-hydroxybutyryl CoA to 4-hydroxybutyraldehyde.

In the butyraldehyde dehydrogenase, Gly226 of SEQ ID NO: 1 may be substituted with another amino acid. This other amino acid may be an amino acid selected from the group consisting of Ile, Leu, Phe, and Tyr. In addition, in the butyraldehyde dehydrogenase, Met227 of SEQ ID NO: 1 may be substituted with another amino acid. This other amino acid may be an amino acid selected from the group consisting of Ile, Leu, Gln, and Val. In addition, in the butyraldehyde dehydrogenase, Leu-273 of SEQ ID NO: 1 may be substituted with Ile.

In addition, in the butyraldehyde dehydrogenase, Gly226 of SEQ ID NO: 1 may be substituted with one amino acid selected from the group consisting of Ile, Leu, Phe, and Tyr, and Met227 of SEQ ID NO: 1 may be substituted with an amino acid selected from the group consisting of Ile, Leu, Gin, and Val.

In the butyraldehyde dehydrogenase, Gly226 of SEQ ID NO: 1 may be substituted with one amino acid selected from the group consisting of Ile, Leu, Phe, and Tyr, and Leu273 of SEQ ID NO: 1 may be substituted with Ile.

In addition, in the butyraldehyde dehydrogenase, Leu273 of SEQ ID NO: 1 may be substituted with Ile, and Met-227 of SEQ ID NO: 1 may be substituted with an amino acid selected from the group consisting of Ile, Leu, Gin, and Val.

In addition, in the butyraldehyde dehydrogenase, Gly226 of SEQ ID NO: 1 may be substituted with one amino acid selected from the group consisting of Ile, Leu, Phe, and Tyr, Met227 of SEQ ID NO: 1 may be substituted with an amino acid selected from the group consisting of Ile, Leu, Gin, and Val, and Leu-273 may be substituted with Ile.

In SEQ ID NO: 1, the 226th, 227th, and 273th amino acids or a combination thereof may be a binding site of the butyraldehyde dehydrogenase to NADH or NADPH.

Another aspect of the present invention provides a polynucleotide encoding the butyraldehyde dehydrogenase mutant.

In this description, the term “polynucleotide” generally includes DNA and RNA molecules such as gDNA and cDNA, and a nucleotide which is a basic unit of a polynucleotide may include not only a natural nucleotide but also an analogue wherein a sugar or a base part is modified. The polynucleotide may be an isolated polynucleotide. A polynucleotide encoding the butyraldehyde dehydrogenase may be derived from Clostridium saccharoperbutylacetonicum. The polynucleotide may have an amino acid sequence of any of SEQ ID NO: 9 to SEQ ID NO: 14.

Another aspect of the present invention provides a vector including the polynucleotide having the nucleotide sequence encoding the butyraldehyde dehydrogenase mutant. The polynucleotide may be operably connected to a regulatory sequence. The regulatory sequence may include a promoter, a terminator or an enhancer. In addition, the promoter may be operably bound to a sequence encoding a gene. In this description, the term “operably connected” may refer to a functional connection between a nucleic acid expression regulatory sequence and another nucleotide sequence. Due to the operable connection, the regulatory sequence may regulate transcription and/or translation of a nucleotide encoding the butyraldehyde dehydrogenase mutant.

Another aspect of the present invention provides a microorganism including a polynucleotide encoding the butyraldehyde dehydrogenase having a catalytic activity of converting 4-hydroxybutyryl CoA to 4-hydroxybutyraldehyde.

The microorganism may include a non-natural or recombinant microorganism. The term “non-natural” means having at least one genetic modification which is not generally found in a natural strain of a reference species including a wild-type strain of the reference species. Genetic alterations include, for example, modifications introducing expressible nucleic acid encoding metabolic polypeptides, other nucleic acid additions, nucleic acid deletions and/or other functional disruption of the microbial organism's genetic material. The modifications include an encoding part and a functional fragment thereof with respect to heterogenous, homogenous, or both heterogenous and homogenous polypeptides of the reference species. An additional modification includes, for example, a non-coding regulatory part wherein the modification alters expression of a gene or an operon. An example of the metabolic polypeptide includes an enzyme or a protein in a biological synthetic pathway of butanediols such as 4-HB or 1,4-butanediol. Therefore, a non-natural microorganism may include a genetic modification to a nucleic acid encoding a metabolic polypeptide or a functional fragment thereof.

The microorganism may express the butyraldehyde dehydrogenase.

The microorganism may have an increased catalytic activity of converting 4-hydroxybutyryl CoA to 4-hydroxybutyraldehyde and/or 1,4-butanediol. The catalytic activity of converting 4-hydroxybutyryl CoA to 4-hydroxybutyraldehyde and/or 1,4-butanediol may have been increased to a sufficient degree to produce 4-hydroxybutyraldehyde or 1,4-butanediol. The activity may have been increased by about 110%, about 120%, about 130%, about 140%, about 150%, about 160%, about 170%, about 200%, about 400%, about 500%, about 1000%, about 2000% or about 10,000% or more with reference to the activity of a control group (e.g., a non-recombinant microorganism).

In addition, the microorganism may have an increased catalytic activity of converting 4-hydroxybutyrate to 4-hydroxybutyryl CoA, and/or converting succinyl CoA, alpha-ketoglutarate or a combination thereof to 4-hydroxybutyrate. The catalytic activity may have been increased to a sufficient degree to produce 4-hydroxybutyraldehyde and/or 1,4-butanediol. The activity may have been increased by about 110%, about 120%, about 130%, about 140%, about 150%, about 160%, about 170%, about 200%, about 400%, about 500%, about 1000%, about 2000% or about 10,000% or more with reference to that of a control group (e.g., a non-recombinant microorganism).

In the microorganism, conversion to 4-hydroxybutyryl CoA may have been increased by increasing an expression of a polypeptide catalyzing a conversion of 4-hydroxybutyrate to 4-hydroxybutyryl CoA. The polypeptide may be 4-hydroxybutyryl-CoA transferase (Cat2). The enzyme may be an enzyme classified as EC 2.8.3.-. An increase of the enzyme may occur due to an increase of an endogenous gene or an introduction of an exogenous gene. An increase of an endogenous gene may occur due to a gene amplification or a mutation of a regulatory domain. The exogenous gene may be a homogenous or a heterogenous gene. The microorganism may have an introduced gene encoding a polypeptide catalyzing a conversion of 4-hydroxybutyrate to 4-hydroxybutyryl CoA, for example, an introduced gene encoding 4-hydroxybutyryl CoA-transferase. A polynucleotide encoding 4-hydroxybutyryl CoA-transferase may have been derived from Porphyromonas gingivali.

The microorganism may have an increased activity of converting succinyl CoA, alpha-ketoglutarate or a combination thereof to 4-hydroxybutyrate. The catalytic activity of converting succinyl CoA, alpha-ketoglutarate or a combination thereof to 4-hydroxybutyrate may have been increased by an increased expression of a polypeptide converting succinyl CoA to succinic semialdehyde, a polypeptide converting alpha-ketoglutarate to succinic semialdehyde, a polypeptide converting succinic semialdehyde to 4-hydroxybutyrate or a combination thereof.

A polypeptide converting succinyl CoA to succinic semialdehyde may be CoA-dependent succinate semialdehyde dehydrogenase (SucD). The succinate semialdehyde dehydrogenase may be an enzyme classified as EC.1.2.1. A polypeptide converting alpha-ketoglutarate to succinic semialdehyde may be α-ketoglutarate decarboxylase (SucA). The α-ketoglutarate decarboxylase may be an enzyme classified as EC 4.1.1.71. A polypeptide converting succinic semialdehyde to 4-hydroxybutyrate may be 4-hydroxybutyrate dehydrogenase (4Hbd). The 4-hydroxybutyrate dehydrogenase may be an enzyme classified as EC.1.1.1.-(oxidoreductase with NAD+ or NADP+ as acceptor). The 4-hydroxybutyrate dehydrogenase may be NAD-dependent.

A polypeptide converting succinyl CoA to succinic semialdehyde, a polypeptide converting alpha-ketoglutarate to succinic semialdehyde, and a polypeptide converting succinic semialdehyde to 4-hydroxybutyrate may have the amino acid sequences of SEQ ID NOS: 18, 20, and 22, respectively.

In the microorganism, the catalytic activity of converting succinyl CoA, alpha-ketoglutarate or a combination thereof to 4-hydroxybutyrate may be increased by an introduction of a gene encoding a polypeptide converting succinyl CoA to succinic semialdehyde, a gene encoding a polypeptide converting alpha-ketoglutarate to succinic semialdehyde, a gene encoding a polypeptide converting succinic semialdehyde to 4-hydroxybutyrate or a combination thereof. A gene encoding a polypeptide converting succinyl CoA to succinic semialdehyde, a gene encoding a polypeptide converting alpha-ketoglutarate to succinic semialdehyde, and a gene encoding a polypeptide converting succinic semialdehyde to 4-hydroxybutyrate may have the nucleotide sequences of SEQ ID NOS: 19, 21, and 23, respectively.

In an embodiment of producing 1,4-butanediol, the microorganism can convert a substrate to a product in at least one conversion selected from the group consisting of conversions from succinyl CoA to succinic semialdehyde and/or from alpha-ketoglutarate to succinic semialdehyde; from succinic semialdehyde to 4-hydroxybutyrate; from 4-hydroxybutyrate to 4-hydroxybutyryl CoA; and from 4-hydroxybutyryl CoA to 4-hydroxybutyraldehyde. In addition, the microorganism can convert 4-hydroxybutyraldehyde to 1,4-butanediol.

In the microorganism, an activity of converting pyruvate to lactate, an activity of converting pyruvate to formate, an activity of converting acetyl Co-A to ethanol, an activity of converting oxaloacetate to malate, an activity of regulating aerobic respiration control or a combination thereof may have been eliminated or reduced. The term “reduced” or “reduction” may represent a relative activity of the mutated microorganism in comparison with the activity of the microorganism that is not mutated (e.g., non-recombinant microorganism). The activity may have been decreased by about 75%, about 80%, about 85%, about 90%, about 95% or about 100% with reference to the activity of a species in an appropriate control group (e.g., non-recombinant microorganism).

In the microorganism, an expression of a polypeptide converting pyruvate to lactate, a polypeptide converting pyruvate to formate, a polypeptide converting acetyl Co-A to ethanol, a polypeptide converting oxaloacetate to malate, a polypeptide regulating aerobic respiration control or a combination thereof may have been eliminated or reduced. In the microorganism, a gene encoding a polypeptide converting pyruvate to lactate, a gene encoding a polypeptide converting pyruvate to formate, a gene encoding a polypeptide converting acetyl Co-A to ethanol, a gene encoding a polypeptide converting oxaloacetate to malate, a gene encoding a polypeptide regulating aerobic respiration control or a combination thereof may have been inactivated or reduced.

A polypeptide converting pyruvate to lactate may be an enzyme classified as EC.1.1.1.27 or EC.1.1.2.3. The polypeptide converting pyruvate to lactate may be derived from an Escherichia. The polypeptide may be derived from an Escherichia W chromosome. A gene encoding the polypeptide converting pyruvate to lactate may have the Gene ID 12753486. The gene may be Escherichia IdhA encoding NADH-linked lactate dehydrogenase.

A polypeptide converting pyruvate to formate may be an enzyme reversibly converting pyruvate to formate. The enzyme may catalyze the reaction, pyruvate+CoA ⇄formate+acetyl CoA. The enzyme may be Escherichia pyruvate formate lyase (Pfl). The pyruvate formate lyase may be an enzyme classified as EC.2.3.1.54. A gene encoding the polypeptide converting pyruvate to formate may have the Gene ID 12752499. The gene may have the nucleotide sequence of SEQ ID NO: 25. The gene may be Escherichia pflB encoding pyruvate formate lyase.

A polypeptide converting acetyl Co-A to ethanol may be alcohol dehydrogenase (Adh). The alcohol dehydrogenase (Adh) may be an enzyme reversibly converting acetyl Co-A to ethanol along with oxidation of NADH to NAD⁺. The alcohol dehydrogenase (Adh) may be an enzyme classified as EC.1.1.1.1. A gene encoding the polypeptide converting acetyl Co-A to ethanol may have the Gene ID 12753141. The gene may have the nucleotide sequence of SEQ ID NO: 26. The gene may be Escherichia adhE encoding NADH-linked alcohol dehydrogenase (Adh).

A polypeptide converting oxaloacetate to malate may be an enzyme catalyzing a conversion of oxaloacetate to malate by using reduction of NAD⁺ to NADH. The enzyme may be malate dehydrogenase (Mdh). The malate dehydrogenase (Adh) may be an enzyme classified as EC 1.1.1.37. The malate dehydrogenase (Adh) may have the amino acid sequence of SEQ ID NO: 27. The gene encoding the malate dehydrogenase (Adh) may have the nucleotide sequence of SEQ ID NO: 28.

A polypeptide of a factor regulating aerobic respiration control may be aerobic respiration control A (ArcA). The ArcA may be a DNA-binding response regulator. The ArcA be a DNA-binding response regulator of a two-component system. The ArcA may belong to a two-component (ArcB-ArcA) signal-transduction system group, and form a global regulation system controlling positively or negatively expression of various operons in cooperation with an isologous sensory kinase. The ArcA may induce expression of gene products allowing for activation of sensitive central metabolic enzymes at a low oxygen level by acting under microaerobic conditions. Deletion of arcA/arcB under microaerobic conditions may increase inactivation of ldh, icd, gltA, mdh, and gdh genes. The ArcA may have the amino acid sequence of SEQ ID NO: 29. The ArcA may have the nucleotide sequence of SEQ ID NO: 30.

The microorganism may express a mutant of an exogenous pyruvate dehydrogenase subunit, a mutant of an NADH insensitive citrate synthase or a combination thereof.

An exogenous pyruvate dehydrogenase subunit may be derived from Klebsiella pneumonia. The pyruvate dehydrogenase subunit may be LpdA. The Klebsiella pneumonia-derived LpdA may have the amino acid sequence of SEQ ID NO: 33. An expression of the exogenous pyruvate dehydrogenase subunit may be increased by an introduction of an exogenous gene. The exogenous gene may be Klebsiella pneumonia-derived lpdA and have the nucleotide sequence of SEQ ID NO: 34. In a mutant of the exogenous pyruvate dehydrogenase subunit, Glu354 may be substituted with another amino acid in SEQ ID NO: 33. This other amino acid may be Lys. The microorganism may include a polynucleotide encoding a mutant of the exogenous pyruvate dehydrogenase subunit. The polynucleotide may have the nucleotide sequence of SEQ ID NO: 36.

A NADH insensitive citrate synthase may be GltA. The GltA may have the amino acid sequence of SEQ ID NO: 37. A mutant of the NADH insensitive citrate synthase may be formed by substituting Arg146 of SEQ ID NO: 37 with another amino acid. The mutant may have the amino acid sequence of SEQ ID NO: 39. This other amino acid may be Leu. The microorganism may include a polynucleotide encoding a mutant of the NADH insensitive citrate synthase. The polynucleotide may have the nucleotide sequence of SEQ ID NO: 40.

In the microorganism, a polynucleotide encoding a mutant of pyruvate dehydrogenase may be included; the activity of converting 4-hydroxybutyrate to 4-hydroxybutyryl CoA may be increased; the activity of converting succinyl CoA and/or alpha-ketoglutarate to 4-hydroxybutyrate may be increased; a gene encoding lactate dehydrogenase converting pyruvate to lactate, a gene encoding pyruvate formate lyase converting pyruvate to formate, a gene encoding alcohol dehydrogenase converting acetyl-CoA to ethanol, a gene encoding a polypeptide converting oxaloacetate to malate, a gene encoding a factor regulating aerobic respiration control or a combination thereof may be inactivated or reduced; an exogenous pyruvate dehydrogenase subunit, a mutant of an NADH insensitive citrate synthase or a combination thereof may be expressed; the activity of converting 4-hydroxybutyrate to 4-hydroxybutyryl CoA may be increased because a gene encoding a polypeptide converting 4-hydroxybutyrate to 4-hydroxybutyryl CoA has been introduced, the activity of converting succinyl CoA and/or alpha-ketoglutarate to 4-hydroxybutyrate may be increased because a gene encoding a polypeptide converting succinyl CoA to succinic semialdehyde, a gene encoding a polypeptide converting alpha-ketoglutarate to succinic semialdehyde, a gene encoding a polypeptide converting succinic semialdehyde to 4-hydroxybutyrate or a combination thereof has been introduced; an exogenous pyruvate dehydrogenase subunit may be included because a gene encoding Klebsiella pneumonia-derived lpdA, and a gene encoding a mutant of the NADH insensitive citrate synthase have been introduced.

The microorganism may include a vector including a polynucleotide having a nucleotide encoding the butyraldehyde dehydrogenase mutant. In the microorganism, the vector may have been introduced. The introduction may involve transformation.

The introduction of a gene may be any type of introduction, and may be, for example, an introduction in the form of an expression cassette, an introduction of the gene itself or an introduction of a polynucleotide structure. The expression cassette may include all factors related to the expression of the gene by itself. The expression cassette may a polynucleotide structure. The expression cassette may include a promoter, a transcription termination signal, a ribosome binding site and a translation termination signals operably connected with the gene. The expression cassette may be an expression vector capable of self-replication. An introduction of the expression cassette or an introduction in the form of a polynucleotide structure may be operably connected with a sequence related to an expression in the host cell to which the expression cassette or the polynucleotide structure is introduced.

The term “transformation” used herein may refer to introducing a gene to a host cell so that the gene may be expressed in the host cell. A transformed gene may be inserted in a chromosome of a host cell and/or located outside the chromosome. The gene includes DNA or RNA.

The microorganism may mean an arbitrary organism existing as a microscopic cell included in Archaea, bacteria or eukaryote domains. The microorganism may include a prokaryote or a eukaryote or an organism of a microscopic size. The microorganism may include not only a eukaryote such as yeast and fungus but also all species of bacteria, Archaea, and eubacteria. In addition, the microorganism may include an arbitrary cell culture of an arbitrary species which may be cultured for biological production.

The microorganism may be of Escherichia genus. The Escherichia genus microorganism may include Escherichia coli, Escherichia albertii, Escherichia blattae, Escherichia fergusonii, Escherichia hermannii or Escherichia vulneris.

Another aspect of the present invention provides a method of producing 4-hydroxybutyraldehyde including a step wherein 4-hydroxybutyryl CoA is contacted with a butyraldehyde dehydrogenase mutant.

The contact may include culturing. The culturing may be performed in a culture medium including the butyraldehyde dehydrogenase mutant and 4-hydroxybutyryl CoA. In the method, the butyraldehyde dehydrogenase mutant is the same as the mutant described above.

Another aspect of the present invention provides a method of producing 1,4-butanediol by culturing of a microorganism expressing a butyraldehyde dehydrogenase mutant including a step wherein 1,4-butanediol is produced in the culture; and a step wherein the 1,4-butanediol is recovered from the culture.

The microorganism is the same microorganism described above. The culturing may be fermentation. The fermentation may be fed-batch fermentation and batch separation, fed-batch fermentation and continuous separation, or continuous fermentation and continuous separation.

The culturing of the microorganism may be performed in an appropriate culture medium and according to an appropriate culture condition known to the concerned industry. The culturing procedure may be conveniently adjusted according to the selected microorganism. The culturing method may include at least one culturing method selected from the group consisting of batch culturing, continuous culturing, and fed-batch culturing.

The culture medium used for the culturing may satisfy requirements of a particular microorganism. The culture medium may include a carbon source, a nitrogen source, trace elements or a combination thereof.

The carbon source may be carbohydrate, lipid, fatty acid, alcohol, organic acid or a combination thereof. The carbohydrate may be glucose, sucrose, lactose, fructose, maltose, starch, cellulose or a combination thereof. The lipid may be soybean oil, sunflower oil, castor oil, coconut oil or a combination thereof. The fatty acid may be palmitic acid, stearic acid, linoleic acid or a combination thereof. The alcohol may be glycerol or ethanol. The organic acid may include acetic acid.

The nitrogen source may include an organic nitrogen source, an inorganic nitrogen source or a combination thereof. The organic nitrogen source may be peptone, yeast extract, meat extract, malt extract, corn steep liquid, soybean meal or a combination thereof. The inorganic nitrogen source may be urea, ammonium sulfate, ammonium chloride, ammonium phosphate, ammonium carbonate, ammonium nitrate or a combination thereof.

The culture medium may include phosphorus, metal salts, amino acids, vitamins, precursors or a combination thereof. The phosphorus source may include potassium dihydrogen phosphate, dipotassium phosphate or a sodium-containing salt corresponding to potassium dihydrogen phosphate and dipotassium phosphate. The metal salt may be magnesium sulfate or iron sulfate.

The culture medium or an individual component may be added to batch culturing, continuous culturing and fed-batch culturing.

In the culturing method, the pH of the culture may be adjusted. The adjustment of the pH may be performed by adding ammonium hydroxide, potassium hydroxide, ammonia, phosphoric acid or sulfuric acid to the culture. In addition, the culturing method may include repression of bubble formation. The repression of bubble formation may be performed by using an endoplasmic reticulum. The endoplasmic reticulum may include fatty acid polyglycol ester. In addition, the step of culturing the microorganism may be performed under substantially anaerobic conditions. The term “substantial anaerobic conditions” means, when the term is used in relation to culture or growth conditions, that the quantity of oxygen in a liquid medium is less than about 10% of the dissolved oxygen saturation. In addition, the substantial anaerobic conditions may include a sealed chamber of a liquid or solid medium maintained in oxygen atmosphere less than about 1% oxygen.

In the culturing, the temperature of the culture may be between about 20 to about 45° C., for example, about 22 to about 42° C., or about 25 to about 45° C. The culture duration may be extended until a desired amount of 1,4-butanediol production is acquired.

Hereinafter, the embodiments of the present invention are described in detail with reference to Examples, but the embodiments of the present invention are not limited thereto.

### Example 1

### Preparation of a Mutant Microorganism Enabling Effective Production of 1,4-butanediol

In Example 1 below, in Escherichia W (ATCC 9637), a gene encoding an enzyme involved in the synthetic pathway of lactate, a major byproduct under anaerobic conditions (IdhA, SEQ ID NO: 24), a gene encoding an enzyme involved in the synthetic pathway of formate (pflB, SEQ ID NO: 25), a gene encoding an enzyme involved in the synthetic pathway of ethanol (adhE, SEQ ID NO: 26), and a gene encoding an enzyme involved in the synthetic pathway of succinate (mdh, SEQ ID NO: 28) were deleted. In addition, to activate the tricarboxylic acid cycle, which is a central metabolic pathway for the purpose of strengthening cell growth and carbon source (glucose) consumption under anaerobic conditions, lpdA (SEQ ID NO: 32), which is one of the genes encoding an enzyme converting pyruvate to acetyl-CoA, was substituted with a mutant of Klebsiella pneumonia-derived lpdA gene capable of reducing the effect of anaerobic conditions on activity under anaerobic conditions, a mutant of gltA gene (SEQ ID NO: 40) capable of reducing the effect of anaerobic conditions on activity under anaerobic conditions was introduced instead of gltA which is a gene encoding an enzyme converting Acetyl-CoA to citrate, and arcA gene (SEQ ID NO: 30) encoding an enzyme repressing tricarboxylic acid gene expression under anaerobic conditions was deleted.

A mutant Escherichia W capable of effectively producing 1,4-butanediol was prepared by transforming the mutated Escherichia W with a recombinant vector including a gene encoding 4-hydroxybutyryl-CoA transferase and a gene encoding butyraldehyde dehydrogenase (Bid) or a mutant thereof.

### 1.1 Preparation of a Mutant Microorganism Wherein a Metabolic Pathway is Mutated for Prevention of Byproduct (Lactate, Formate, Ethanol, and Succinate) Production and for Cell Growth and Carbon Source Consumption Under Anaerobic Conditions

1.1.1 Deletion of IdhA, pflB, adhE, mdh, and arcA Genes

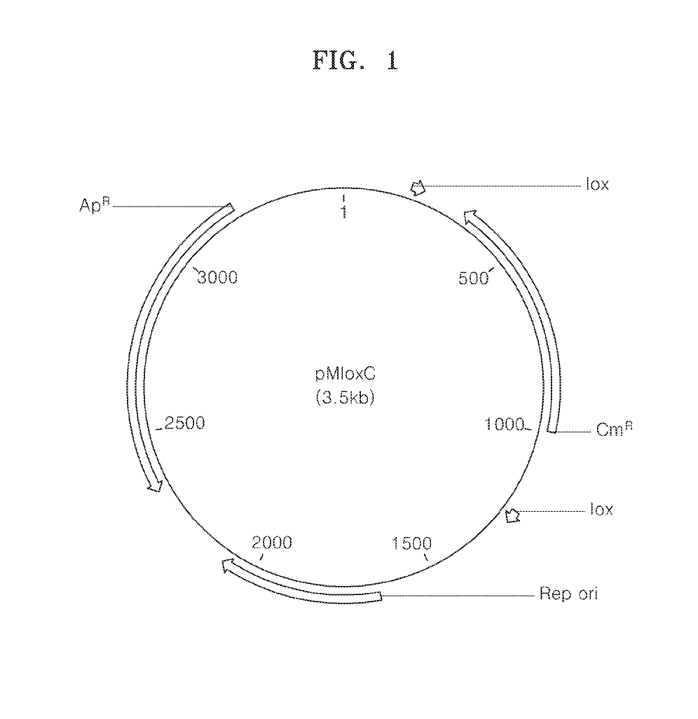

In Escherichia W (ATCC 9637), IdhA, pflB, adhE, mdh, and arcA genes were deleted with a primer below by using a one-step inactivation method (Warner et al., PNAS, 6; 97(12):6640-6645, 2000; Lee, K. H. et al., Molecular Systems Biology 3, 149, 2007). FIG. 1 shows a pMloxC vector map.

To delete the IdhA gene, a polymerase chain reaction (PCR) was performed with the primers of SEQ ID NOS: 41 and 42 using a pMloxC vector as a template. A mutant strain wherein IdhA was deleted was prepared by electroporating the acquired DNA fragments to the competent cell of the W strain wherein λ-red recombinase was expressed. To verify the deletion of the IdhA gene, a colony PCR was performed with the primers of SEQ ID NOS: 51 and 52. As a result, Escherichia W ATCC 9637 (ΔldhA) was obtained.

In addition, the primers of SEQ ID NOS: 43 and 44 were used one by one in the same method described above to delete the pflB gene, and the primers of SEQ ID NOS: 53 and 54 were used to verify the deletion of the pflB gene. As a result, Escherichia W ATCC 9637 (ΔldhAΔpflB) was obtained.

In addition, the primers of SEQ ID NOS: 45 and 46 were used one by one in the same method described above to delete the adhE gene, and the primers of SEQ ID NOS: 55 and 56 were used to verify the deletion of the adhE gene. As a result, Escherichia W ATCC 9637 (ΔldhAΔpflBΔadhE) was obtained.

In addition, the primers of SEQ ID NOS: 47 and 48 were used one by one in the same method described above to delete the mdh gene, and the primers of SEQ ID NOS: 57 and 58 were used to verify the deletion of the mdh gene. As a result, Escherichia W ATCC 9637 (ΔldhAΔpflBΔadhEΔmdh) was obtained.

In addition, the primers of SEQ ID NOS: 49 and 50 were used one by one in the same method described above to delete the arcA gene, and the primers of SEQ ID NOS: 59 and 60 were used to verify the deletion of the arcA gene. As a result, Escherichia W ATCC 9637 (ΔldhAΔpflBΔadhEΔmdhΔarcA) was obtained.

1.1.2 Substitution of Original Escherichia W lpdA Gene with a Klebsiella Pneumonia-Derived lpdA Gene Mutant

In a Escherichia W (ATCC 9637) (ΔldhAΔpflBΔadhEΔmdhΔarcA) strain, the original Escherichia W lpdA gene was substituted with a Klebsiella pneumonia-derived lpdA gene mutant with the primers below by using the one-step inactivation method (Warner et al., PNAS, 6; 97(12):6640-6645, 2000; Lee, K. H. et al., Molecular Systems Biology 3, 149, 2007).

The Klebsiella pneumonia-derived lpdA gene mutant was acquired through site-directed mutagenesis using the primers SEQ ID NOS: 69 and 70. To substitute the original Escherichia W lpd gene with the Klebsiella pneumonia-derived lpdA gene mutant, a PCR was performed with the primers of SEQ ID NOS: 71 and 72 using a pMloxC vector as a template. The lpd gene was substituted with a sacB-Km cassette by electroporating the acquired DNA fragments to the competent cell of the W strain wherein λ-red recombinase was expressed.

Then, the part wherein the lpd gene had been substituted with the sacB-Km cassette was substituted with the Klebsiella pneumonia-derived lpdA gene mutant by performing the one-step inactivation method (Warner et al., PNAS, 6; 97(12):6640-6645, 2000; lee, K. H. et al., Molecular Systems Biology 3, 149, 2007) once again by performing a PCR with the primers of SEQ ID NOS: 73 and 74 using a pMloxC vector as a template. To verify the substituted gene, a colony PCR was performed with the primers of SEQ ID NOS: 75 and 76. As a result, Escherichia W (ATCC 9637) (ΔldhAΔpflBΔadhE ΔlpdA::K.lpdA(E354K)ΔmdhΔarcA) was obtained.

1.1.3 Introduction of a Mutant of Original Escherichia W gltA Gene

In the Escherichia W (ATCC 9637) (ΔldhAΔpflBΔadhEΔmdhΔarcA::K.lpdA (E354K)) strain, a mutant of the original Escherichia W gltA gene was introduced by using the one step inactivation method (Warner et al., PNAS, 6; 97(12):6640-6645, 2000; Lee, K. H. et al., Molecular systems biology 3, 149, 2007) with the primers below.

The mutant of the original Escherichia W gltA gene was prepared by site-directed mutagenesis using the primers of SEQ ID NOS: 77 and 78. To substitute the original Escherichia W gltA gene with gltA (R164L), a PCR was performed with the primers of SEQ ID NOS: 79 and 80 using a pMloxC vector as a template. The gltA gene was substituted with the sacB-Km cassette by electroporating the acquired DNA fragments to the competent cell of the W strain wherein λ-red recombinase was expressed. Then, the part wherein the gltA gene had been substituted with the sacB-Km cassette was finally substituted with gltA (R164L) by performing the one-step inactivation method (Warner et al., PNAS, 6; 97(12):6640-6645, 2000; Lee, K. H. et al., Molecular Systems Biology 3, 149, 2007) once again by performing a PCR with the primers of SEQ ID NOS: 81 and 82 using the pMloxC vector as a template. The chromosomal DNA of the Escherichia W-derived mutant strain prepared by the method described above is Escherichia W (ATCC 9637) (ΔldhAΔpflBΔadhEΔlpdA::K.lpdA (E354K) ΔmdhΔarcA gltA (R164L)).

### 1.2 Introduction of a Gene Encoding a Butyraldehyde Dehydrogenase Mutant and Cat2 Gene

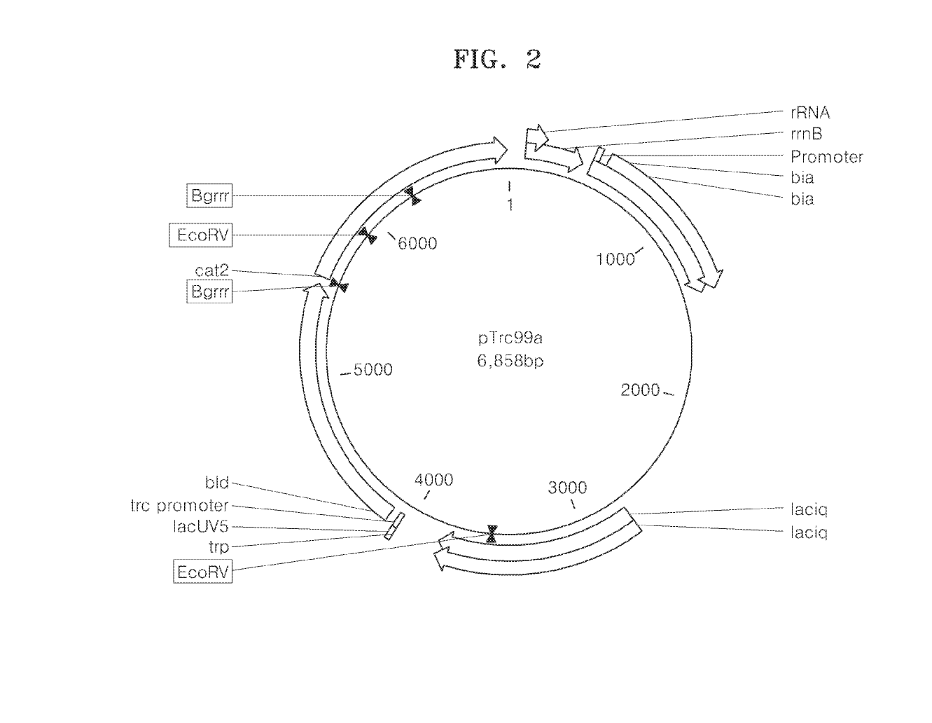

A Porphyromonas gingivali-derived cat2 gene of SEQ ID NO: 17 was synthesized. The pTrc99a (cat2) gene was prepared by introducing the obtained cat2 gene to pTrc99a (Invitrogen) by using the restriction enzymes, EcoRI and HindIII. FIG. 2 shows a pTrc99a vector.

The wild-type butyraldehyde dehydrogenase gene of SEQ ID NO: 2 was amplified by performing a PCR with the primers of SEQ ID NOS: 61 and 62 by using the gDNA of Clostridium saccharoperbutylacetonicum as a template. pTrc99a (bld_{wt}) was prepared by introducing the acquired gene encoding wild-type butyraldehyde dehydrogenase to pTrc99a by using the restriction enzyme, NcoI/EcoRI.

In addition, the gene (bld_{M}) encoding a butyraldehyde dehydrogenase mutant was acquired by performing site-directed mutagenesis by using the acquired wild-type butyraldehyde dehydrogenase gene as a template. The SEQ ID Numbers of the mutant genes are SEQ ID NOS: 10 to 16. Table 1 shows the information about the butyraldehyde dehydrogenase mutants. Each of the pTrc99a (bld_{M}) genes was prepared by respectively introducing bldH, bldI, bldJ, bldL, bldS2, bldS, and bld (G226L), the genes encoding a butyraldehyde dehydrogenase mutant, to pTrc99a by using the restriction enzyme, NcoI/EcoRI.

TABLE 1

| | Butyraldehyde | | | DNA |
|---|---|---|---|---|
| | dehydrogenase | | Protein SEQ | SEQ ID |
| No. | mutants (Bld_{M}) | Mutation | ID NO: | NO: |
| 1 | BldH | M227I | 3 | 10 |
| 2 | BldI | M227L | 4 | 11 |
| 3 | BldJ | M227Q | 5 | 12 |
| 4 | BldL | M227V | 6 | 13 |
| 5 | BldS2 | L273I | 7 | 14 |
| 6 | BldS | M227L + L273I | 8 | 15 |
| 7 | Bld(G226L) | G226L | 9 | 16 |

pTrc99a (bld_{M}) and pTrc99a (cat2) were respectively introduced to Escherichia W (ATCC 9637) (ΔldhAΔpflBΔadhEΔmdhΔarcA) by infusion cloning. The Escherichia W (ATCC 9637) (ΔldhAΔpflBΔadhEΔmdhΔarcA) wherein pTrc99a (bld_{M}) and pTrc99a (cat2) had been introduced were verified and selected. As a result, the Escherichia W (ATCC 9637) (ΔldhAΔpflBΔadhEΔmdhΔarcA) wherein pTrc99a (bld_{M}) and pTrc99a (cat2) were introduced was obtained.

In addition, pTrc99a (bld_{wt}) and pTrc99a (cat2) were respectively introduced to Escherichia W (ATCC 9637) (ΔldhAΔpflBΔadhEΔmdhΔarcA) infusion cloning. The Escherichia W (ATCC 9637) (ΔldhAΔpflBΔadhEΔmdhΔarcA) wherein pTrc99a (bld_{wt}) and pTrc99a (cat2) had been introduced were verified and selected. As a result, the Escherichia W (ATCC 9637) (ΔldhAΔpflBΔadhEΔmdhΔarcA) wherein pTrc99a (bld_{wt}) and pTrc99a (cat2) were introduced was obtained.

1.3 Verification of the Catalytic Activity of Converting 4-Hydroxybutyryl-CoA to 4-Hydroxybutyraldehyde Using the Escherichia W (ATCC 9637) (ΔldhAΔpflBΔadhEΔmdhΔarcA) wherein pTrc99 (BldM-cat2) Had been Introduced

To verify the catalytic activity of the butyraldehyde dehydrogenase mutant of converting 4-hydroxybutyryl-CoA to 4-hydroxybutyraldehyde, the 1,4-BDO production of the Escherichia W (ATCC 9637) (ΔldhAΔpflBΔadhEΔmdhΔarcA) wherein pTrc99a (bld_{M}) and pTrc99a (cat2) had been introduced was measured.

The 1,4-BDO production was compared with the 1,4-BDO production of the control group including the Escherichia W (ATCC 9637) (ΔldhAΔpflBΔadhEΔmdhΔarcA) wherein pTrc99a (bld_{wt}) and pTrc99a (cat2) had been introduced. All other conditions except the mutation of the butyraldehyde dehydrogenase gene were the same in the control group.

The 1,4-BDO production was measured after culturing the Escherichia W (ATCC 9637) (ΔldhAΔpflBΔadhEΔmdhΔarcA) wherein pTrc99a (bld_{M}) and pTrc99a (cat2) had been introduced and the control group under the culture conditions below. The culture was performed under anaerobic conditions, by injecting nitrogen, at 30° C. and 250 rpm for 18 hours. The culture medium was 1 L LB medium including 2% glucose and about 10 mM 4-hydroxybutyrate wherein 100 μg/ml ampicillin and 50 μg/ml kanamycin were added. The pH of the culture medium was adjusted with 5 N NaOH. The strain was cultured until the OD of the culture medium reached 0.4.

The produced 1,4-BDO was analyzed in the following method: 1 ml was taken from 100 ml culture medium, and centrifuged at 13000 rpm for 30 minutes. The supernatant was centrifuged once again under the same conditions, and the sample was prepared by filtering 800 μl of the supernatant with a 0.45 um filter; 10 μl of the sample was analyzed by UHPLC (Ultra High Performance Liquid Chromatography, Water) to measure the quantity of 1,4-BDO; The used UHPLC was Agilent 1100 equipment employing a refractive index detector (RID); 4 mM H₂SO₄ solution was used as a mobile phase, and a BIO-RAD Aminex HPX-87H Column was used as a stationary phase at a flow rate of 0.7 ml/min; The temperature of both the column and the detector was 50° C.

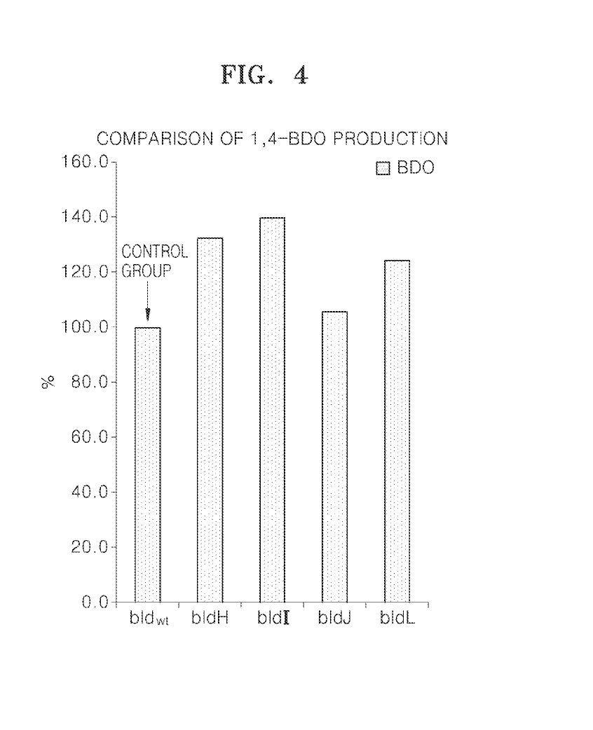

FIG. 4 shows a graph comparing the 1,4-butanediol production of the strains expressing a wild-type butyraldehyde dehydrogenase and butyraldehyde dehydrogenase mutants of BldH, BldI, BldJ or BldL, respectively. As shown in FIG. 4, the 1,4-butanediol production of the strains wherein genes encoding BldH, BldI, BldJ or BldL had been introduced was higher than the 1,4-butanediol production of the control group. This result indicates that the catalytic activity of BldH, BldI, BldJ or BldL of converting 4-hydroxybutyryl-CoA to 4-hydroxybutyraldehyde was excellent and thus the 1,4-butanediol production was increased.

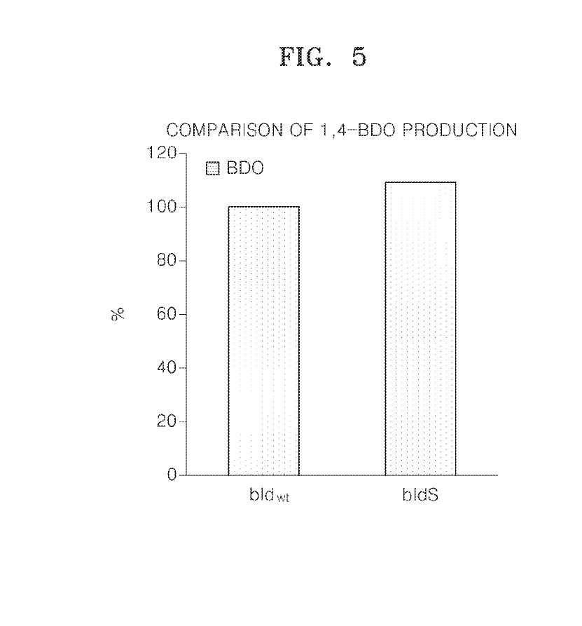

FIG. 5 shows a graph comparing the 1,4-butanediol production of the strains expressing a wild-type butyraldehyde dehydrogenase and a butyraldehyde dehydrogenase mutant of BldS2. As shown in FIG. 5, the 1,4-butanediol production of the strains wherein a gene encoding BldS2 had been introduced was higher than the 1,4-butanediol production of the control group. This result indicates that the catalytic activity of BldS2 of converting 4-hydroxybutyryl-CoA to 4-hydroxybutyraldehyde was excellent and thus the 1,4-butanediol production was increased.

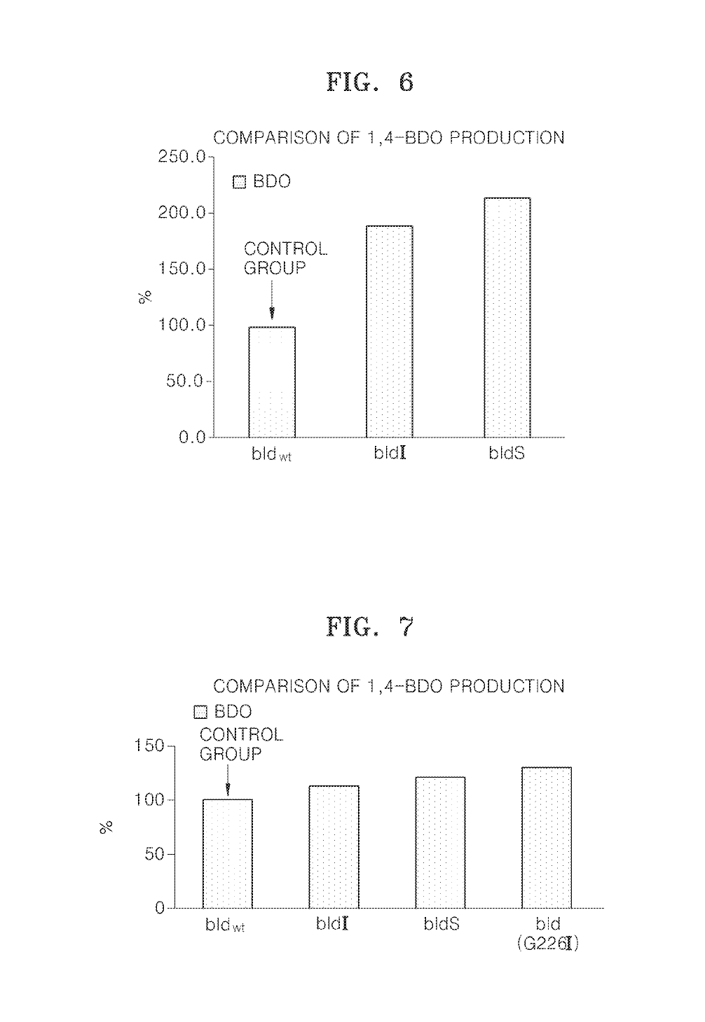

FIG. 6 shows a graph comparing the 1,4-butanediol production of the strains expressing a butyraldehyde dehydrogenase mutant of BldI or BldS, which is a strain wherein pTrc99a (bidI) and pTrc99a (cat2) had been introduced Escherichia (ATCC 9637) (ΔldhAΔpflBΔadhEΔmdhΔarcA) or a strain wherein pTrc99a (bldS) and pTrc99a (cat2) had been introduced Escherichia (ATCC 9637) (ΔldhAΔpflBΔadhEΔmdhΔarcA). As shown in FIG. 6, the 1,4-butanediol production of the strains wherein a gene encoding BldI or BldS had been introduced was higher than the 1,4-butanediol production of the control group. This result indicates that the strain wherein a gene encoding BldI or BldS had been introduced expressed BldI and BldS, the catalytic activity of the butyraldehyde dehydrogenase mutant of converting 4-hydroxybutyryl-CoA to 4-hydroxybutyraldehyde was excellent and thus the 1,4-butanediol production was increased.

### Example 2

### Verification of the 1,4-Butanediol Production by the Butyraldehyde Dehydrogenase Mutant

2.1. Introduction of sucD, 4Hbd and sucA Gene

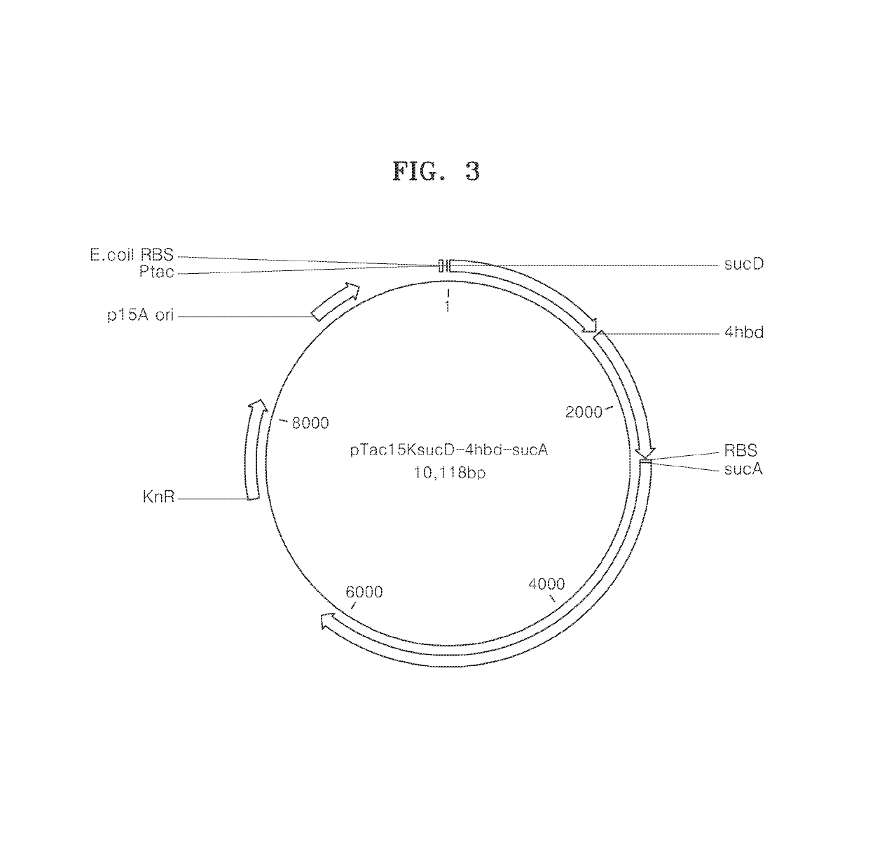

The sucD, 4hbd and sucA genes were introduced to the Escherichia W (ATCC 9637) (ΔldhAΔpflBΔadhEΔmdhΔarcA) prepared in Example 1. The gene of SEQ ID NO: 19 was amplified by performing a PCR with the primer sequences of SEQ ID NOS: 53 and 54 by using the gDNA of Clostridium kluyveri as a template. In addition, the gene of SEQ ID NO: 23 was amplified by performing a PCR with the primer sequences of SEQ ID NOS: 55 and 56 by using the gDNA of Porphyromonas gingivalis as a template. In addition, the gene of SEQ ID NO: 21 was amplified by performing a PCR with the primer sequences of SEQ ID NOS: 57 and 58 by using the gDNA of Mycobacterium bovis as a template. FIG. 3 shows a pTac15k vector.

The pTac15k (sucD) vector was prepared by introducing the sucD gene to pTac15k by using the restriction enzyme EcoRI/Enzyme site. In addition, a pTac15k (4hbd) vector was prepared by introducing the 4hbd gene to pTac15k by using the restriction enzyme EcoRI/Enzyme site and EcoRI/BamHI site. In addition, a pTac15k (sucA) vector was prepared by introducing the sucA gene to pTac15k by using the restriction enzyme EcoRI/SaII site.

The pTac15k (sucD), pTac15k (4hbd), and pTac15k (sucA) vectors were respectively introduced to Escherichia W (ATCC 9637) (ΔldhAΔpflBΔadhEΔmdhΔarcA bld_{M}+cat2+) and Escherichia W (ATCC 9637) (ΔldhAΔpflBΔadhEΔmdhΔarcA bld_{wt}+cat2+) by infusion cloning. The Escherichia W (ATCC 9637) (ΔldhAΔpflBΔadhEΔmdhΔarcA bld_{M}+cat2+) and Escherichia W (ATCC 9637) (ΔldhAΔpflBΔadhEΔmdhΔarcA bld_{wt}+cat2+) wherein the pTac15k (sucD), pTac15k (4hbd), and pTac15k (sucA) vectors had been introduced were acquired by verifying and selecting the same.

2.2 Verification of the 1,4-Butanediol Production by Escherichia W (ATCC 9637) (ΔldhAΔpflBΔadhEΔmdhΔarcA bldM+cat2+) wherein the pTac15k (sucD), pTac15k (4hbd), and pTac15k (sucA) Vectors Had been Introduced

The 1,4-BDO production was measured after culturing the Escherichia W (ATCC 9637) (ΔldhAΔpflBΔadhEΔmdhΔarcA bldM+cat2+) wherein the pTac15k (sucD), pTac15k (4hbd), and pTac15k (sucA) vectors had been introduced under the culture conditions below. In addition, 1,4-BDO production was compared after culturing the Escherichia W (ATCC 9637) (ΔldhAΔpflBΔadhEΔmdhΔarcA bld_{wt}+cat2+) wherein pTac15k (sucD), pTac15k (4hbd), and pTac15k (sucA) had been introduced as the control group.

The culture was performed under anaerobic conditions, by injecting nitrogen, at 30° C. and 250 rpm for 18 hours. Glucose was used as a carbon source. The culture medium was 1 L LB medium including 2% glucose and about 10 mM 4-hydroxybutyrate wherein 100 μg/ml ampicillin and 50 μg/ml kanamycin were added. The pH of the culture medium was adjusted with 5 N NaOH. The strain was cultured until the OD of the culture medium reached 0.4. The 1,4-BDO production was analyzed in the same method described in Example 1.

FIG. 7 shows a graph comparing the 1,4-butanediol production of the strains expressing a wild-type butyraldehyde dehydrogenase and butyraldehyde dehydrogenase mutants of BldI, BldS or Bld (G226I). As shown in FIG. 7, the 1,4-butanediol production of the strains wherein genes encoding BldI, BldS or Bld (G226I) had been introduced was higher than the 1,4-butanediol production of the control group.

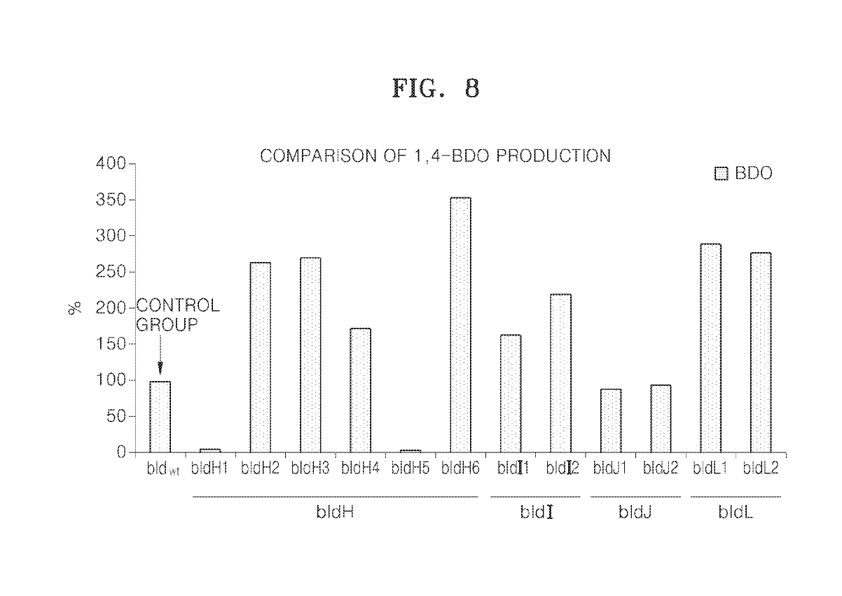

FIG. 8 shows a graph comparing the 1,4-butanediol production of the strains expressing a wild-type butyraldehyde dehydrogenase (Bld_{wt}) and butyraldehyde dehydrogenase mutants of BldH, BldI, BldJ or BldL. The 1,4-butanediol production of the strains expressing butyraldehyde dehydrogenase mutants was verified through multiple colonies including six colonies of the strain expressing BldH, and two colonies of each of the strains expressing BldI, BldJ or BldL. As shown in FIG. 8, the 1,4-butanediol production of the strains wherein genes encoding BldH, BldI, BldJ or BldL had been introduced was higher than the 1,4-butanediol production of the control group.

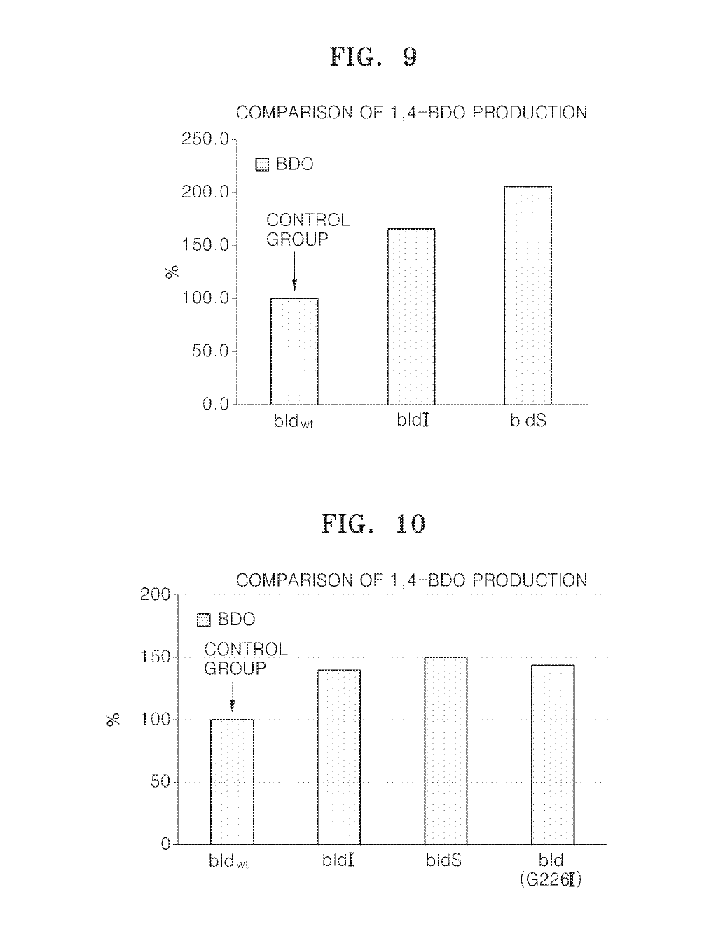

FIG. 9 shows a graph comparing the 1,4-butanediol production of the strains expressing a butyraldehyde dehydrogenase mutant of BldI or BldS, which is a strain wherein pTrc99a (bidI) and pTrc99a (cat2) had been introduced Escherichia (ATCC 9637) (ΔldhAΔpflBΔadhEΔmdhΔarcA) or a strain wherein pTrc99a (bldS) and pTrc99a (cat2) had been introduced Escherichia (ATCC 9637) (ΔldhAΔpflBΔadhEΔmdhΔarcA). As shown in FIG. 9, the 1,4-butanediol production of the strains wherein genes encoding BldI or BldS had been introduced was higher than the 1,4-butanediol production of the control group wherein a wild-type butyraldehyde dehydrogenase was introduced.

FIG. 10 shows a graph comparing the 1,4-butanediol production of the strains expressing a wild-type butyraldehyde dehydrogenase and butyraldehyde dehydrogenase mutants of BldI, BldS or Bld (G226I). As shown in FIG. 10, the 1,4-butanediol production of the strains wherein genes encoding BldI, BldS or Bld (G226I) had been introduced was higher than the 1,4-butanediol production of the control group wherein a wild-type butyraldehyde dehydrogenase was introduced.

These results showed that the strains wherein the mutant genes of the butyraldehyde dehydrogenase gene were introduced expressed butyraldehyde dehydrogenase mutants and produced 1,4-butanediol from glucose, and that the 1,4-butanediol production was higher than the 1,4-butanediol production of the microorganism wherein a wild-type butyraldehyde dehydrogenase was introduced.

As described above, according to an aspect of the present invention, production of 1,4-butanediol may be increased by using a butyraldehyde dehydrogenase mutant, a polynucleotide encoding the same, a vector including the polynucleotide, and a microorganism including the polynucleotide.

Production of 1,4-butanediol may be increased by a method of producing 1,4-butanediol according to an aspect of the present invention.

It should be understood that the exemplary embodiments described herein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A butyraldehyde dehydrogenase that converts 4-hydroxybutyryl CoA to 4-hydroxybutyraldehyde comprising the amino acid sequence of SEQ ID NO: 1 with a mutation of at least one amino acid residue at an NADH or NADPH binding site.

2. The butyraldehyde dehydrogenase of claim 1, wherein one or more of Gly226, Met227, or Leu273 of SEQ ID NO: 1 is substituted with another amino acid.

3. The butyraldehyde dehydrogenase of claim 2, wherein Gly226 of SEQ ID NO: 1 is substituted with Ile, Leu, Phe, or Tyr.

4. The butyraldehyde dehydrogenase of claim 2, wherein Met227 of SEQ ID NO: 1 is substituted with Ile, Leu, Gln, or Val.

5. The butyraldehyde dehydrogenase of claim 2, wherein Leu273 of SEQ ID NO: 1 is substituted with Ile.

6. The butyraldehyde dehydrogenase of claim 2, wherein Leu273 of SEQ ID NO: 1 is substituted with Ile and Met227 of SEQ ID NO: 1 is substituted with Ile, Leu, Gln, or Val.

7. The butyraldehyde dehydrogenase of claim 1, wherein the NADH or NADPH binding site comprises the 226^{th}, 227^{th}, and 273^{th} amino acid residues of SEQ ID NO: 1.

8. The butyraldehyde dehydrogenase of claim 1, comprising a polypeptide selected from the group consisting of SEQ ID NO: 3 to SEQ ID NO: 9.

9. A recombinant microorganism comprising a polynucleotide encoding the butyraldehyde dehydrogenase of claim 1.

10. The recombinant microorganism of claim 9, wherein the microorganism converts 4-hydroxybutyryl CoA to 4-hydroxybutyraldehyde at an increased level relative to a non-recombinant microorganism.

11. The recombinant microorganism of claim 9, wherein the microorganism further comprises a polynucleotide encoding a polypeptide that catalyzes the conversion of succinyl CoA to succinic semialdehyde, a polypeptide that catalyzes the conversion of alpha-ketoglutarate to succinic semialdehyde, a polypeptide that catalyzes the conversion of succinic semialdehyde to 4-hydroxybutyrate, or a combination thereof.

12. The recombinant microorganism of claim 11, wherein the microorganism converts succinyl CoA, alpha-ketoglutarate, or a combination thereof to 4-hydroxybutyrate at an increased level relative to a non-recombinant microorganism.

13. The recombinant microorganism of claim 9, wherein the microorganism further comprises an inactivated gene or lacks a gene encoding a polypeptide that converts pyruvate to lactate, a polypeptide that converts pyruvate to formate, a polypeptide that converts acetyl Co-A to ethanol, a polypeptide that converts oxaloacetate to malate, a polypeptide that regulates aerobic respiration control, or a combination thereof.

14. The recombinant microorganism of claim 13, wherein the microorganism converts pyruvate to lactate, converts pyruvate to formate, converts acetyl Co-A to ethanol, converts oxaloacetate to malate, regulates aerobic respiration control, or a combination thereof at a reduced or eliminated level relative to a non-recombinant microorganism.

15. The recombinant microorganism of claim 9, wherein the recombinant microorganism expresses a mutant of an exogenous pyruvate dehydrogenase subunit, a mutant of an NADH insensitive citrate synthase, or a combination thereof.

16. A method of producing 1,4-butanediol, the method comprising culturing the microorganism of claim 10 in a cell culture medium, whereby the microorganism produces 1,4-butanediol; and
recovering the 1,4-butanediol from the culture.
